(19) United States
(12) Patent Application Publication
(10) Pub. No.: US 2026/0158140 A1
(43) Pub. Date: Jun. 11, 2026

(54) METHODS AND MATERIALS FOR IMPROVING T CELL THERAPIES

(71) Applicant: Mayo Foundation for Medical Education and Research, Rochester, MN (US)

(72) Inventors: Saad J. Kenderian, Rochester, MN (US); Mohamad M. Adada, Rochester, MN (US); Michelle J. Cox, Rochester, MN (US); Elizabeth L. Siegler, Rochester, MN (US); Erin E. Tapper, Sioux Falls, SD (US)

(21) Appl. No.: 18/706,967

(22) PCT Filed: Nov. 3, 2022
(86) PCT No.: PCT/US2022/048769

§ 371 Date: May 2, 2024

Related U.S. Application Data
(60) Provisional application No. 63/275,339, filed on Nov. 3, 2021.

(51) Int. Cl. A61K 40/11; A61K 9/00; A61K 31/415; A61K 31/5025; A61K 31/506; A61K 31/519; A61K 35/17; A61K 40/31; A61K 40/42; A61P 35/00

(52) U.S. Cl. CPC A61K 40/11; A61K 9/0053; A61K 31/415; A61K 31/5025; A61K 31/506; A61K 31/519; A61K 35/17; A61K 40/31; A61K 40/4211; A61P 35/00; A61K 2239/48

(57) ABSTRACT

This document provides methods and materials involved in improving T cell therapies (e.g., CAR T cell therapies). For example, methods and materials for using one or more reversible inhibitors of a BTK polypeptide that bind non-covalently to a BTK polypeptide (e.g. vecabrutinib) in combination with an adoptive cell therapy (e.g., a CART cell therapy) to improve one or more functions of a T cell are provided.

### CROSS-REFERENCE TO RELATED APPLICATIONS

This application claims the benefit of U.S. Patent Application Ser. No. 63/275,339, filed on Nov. 3, 2021. The disclosure of the prior application is considered part of (and is incorporated by reference in) the disclosure of this application.

### TECHNICAL FIELD

This document relates to methods and materials involved in improving adoptive T cell therapies such as chimeric antigen receptor (CAR) T cell therapies. For example, this document provides methods and materials for using adoptive T cell therapies (e.g., CAR T cells) in combination with one or more inhibitors of a Bruton's tyrosine kinase (BTK) polypeptide to improve one or more functions of the T cells (e.g., CAR T cells) of the adoptive T cell therapies to treat conditions such as cancer in a mammal (e.g., a human).

### BACKGROUND INFORMATION

CD19-targeted CAR T (CART19) cell therapy has been remarkably successful in treating a subset of patients with hematological malignancies. However, CAR T cell therapy is associated with significant toxicities, including cytokine release syndrome (CRS) and neurotoxicity (NT). Furthermore, the rate of durable responses after CART19 cell therapy is low, and most patients develop resistance to the therapy. One of the predominant mechanisms for CAR T cell resistance is intrinsic T cell dysfunction rendering many blood T cells insufficient to be fit for immunotherapy.

### SUMMARY

This document provides methods and materials involved in improving adoptive T cell therapies (e.g., CAR T cell therapies). For example, one or more reversible inhibitors of a BTK polypeptide that bind non-covalently to a BTK polypeptide (e.g. vecabrutinib, fenebrutinib, and/or pirtobrutinib) can be administered to a mammal (e.g., a human) having cancer in combination with an adoptive cell therapy (e.g., a CART cell therapy) to improve one or more functions of a T cell to treat cancer in a mammal (e.g., a human). For example, one or more reversible BTK inhibitors that bind non-covalently to a BTK polypeptide (e.g. vecabrutinib, fenebrutinib, and/or pirtobrutinib) can be used in combination with a CAR T cell therapy to improve one or more functions of a CAR T cell.

As demonstrated herein, administration of a reversible BTK inhibitor that binds non-covalently to a BTK polypeptide to a mammal in combination with CAR T cell therapy improved CAR T cell functions by, for example, increasing target cell killing, reducing toxicity to the mammal, reducing the occurrence of cytokine release syndrome (CRS), and/or increasing or maintaining the proliferative potential of the CAR T cells. Thus, one or more reversible BTK inhibitors that bind non-covalently to a BTK polypeptide (e.g. vecabrutinib, fenebrutinib, and/or pirtobrutinib) can be incorporated into adoptive T cell therapies (e.g., CART cell therapies) to treat, for example, cancer.

In general, one aspect of this document features methods for improving T cell therapy. The methods can include, or consist essentially of, administering, to a mammal and at a time that is from about 1 day before to about 360 days after the mammal receives an adoptive T cell therapy, a reversible BTK inhibitor that binds non-covalently to a BTK polypeptide, where the level of proliferation of T cells of the adoptive T cell therapy in the mammal is increased after the administering step as compared to the level of proliferation of T cells of an adoptive T cell therapy in a comparable mammal not administered the BTK inhibitor. The level of proliferation can be increased by at least 5 percent. The level of proliferation can be increased by at least 10 percent. The level of proliferation can be increased by at least 25 percent. The mammal can be a human. The mammal can have cancer, and the adoptive T cell therapy can treat the cancer following the administering step. The cancer can be lymphoma, leukemia, hepatocellular carcinoma, bowel cancer, lung cancer, breast cancer, ovarian cancer, melanoma, head and neck cancer, sarcoma, epithelial cancer, mesothelioma, thyroid cancer, brain cancer, or endocrine cancer. The mammal can have lymphoma, and the adoptive T cell therapy can treat the lymphoma following the administering step. The BTK inhibitor can be vecabrutinib, fenebrutinib, pirtobrutinib, ARQ531, XMU-MP3, CB1763, GNE-431, CGI-1746, RN1486, BMS-986142, HBW-3-10, or CG806. The T cells of the adoptive T cell therapy can include a chimeric antigen receptor. The chimeric antigen receptor can target a tumor-associated antigen. The tumor-associated antigen can be CD19. The adoptive T cell therapy can be a CAR T therapy. The adoptive T cell therapy can cause less expression of one or more cytokine polypeptides associated with cytokine release syndrome within the mammal than the level of expression of the one or more cytokine polypeptides within the comparable mammal that received the adoptive T cell therapy in the absence of the administration of the BTK inhibitor. The cytokine polypeptides can be an IL-6 polypeptide, an IL-10 polypeptide, an MIP-β polypeptide, an IP-10 polypeptide, or a TNF-α polypeptide. The BTK inhibitor can be administered to the mammal at a dose from about 10 mg/kg to about 250 mg/kg (e.g., from about 25 mg/kg to about 75 mg/kg). The BTK inhibitor can be administered to the mammal at least once a day. The BTK inhibitor can be administered to the mammal twice a day. The BTK inhibitor can be administered to the mammal within 1 to 2 months of the mammal receiving the T cell therapy.

In another aspect, this document features methods for improving T cell therapy. The methods can include, or consist essentially of, administering, to a mammal and at a time that is from about 1 day before to about 360 days after the mammal receives an adoptive T cell therapy, a reversible BTK inhibitor that binds non-covalently to a BTK polypeptide, where the adoptive T cell therapy is less toxic to the mammal than the level of toxicity in a comparable mammal receiving an adoptive T cell therapy and not administered the BTK inhibitor. The mammal can be a human. The mammal can have cancer, and the adoptive T cell therapy can treat the cancer following the administering step. The cancer can be lymphoma, leukemia, hepatocellular carcinoma, bowel cancer, lung cancer, breast cancer, ovarian cancer, melanoma, head and neck cancer, sarcoma, epithelial cancer, mesothelioma, thyroid cancer, brain cancer, or endocrine cancer. The mammal can have lymphoma, and the adoptive T cell therapy can treat the lymphoma following the administering step. The BTK inhibitor can be vecabrutinib, fenebrutinib, pirtobrutinib, ARQ531, XMU-MP3, CB1763, GNE-431, CGI-1746, RN1486, BMS-986142, HBW-3-10, or CG806. The T cells of the adoptive T cell therapy can include a chimeric antigen receptor. The chimeric antigen receptor can target a tumor-associated antigen. The tumor-associated antigen can be CD19. The adoptive T cell therapy can be a CAR T therapy. The adoptive T cell therapy can cause less expression of one or more cytokine polypeptides associated with cytokine release syndrome within the mammal than the level of expression of the one or more cytokine polypeptides within the comparable mammal that received the adoptive T cell therapy in the absence of the administration of the BTK inhibitor. The cytokine polypeptides can be an IL-6 polypeptide, an IL-10 polypeptide, an MIP-β polypeptide, an IP-10 polypeptide, or a TNF-α polypeptide. The BTK inhibitor can be administered to the mammal at a dose from about 10 mg/kg to about 250 mg/kg (e.g., from about 25 mg/kg to about 75 mg/kg). The BTK inhibitor can be administered to the mammal at least once a day. The BTK inhibitor can be administered to the mammal twice a day. The BTK inhibitor can be administered to the mammal within 1 to 2 months of the mammal receiving the T cell therapy.

In another aspect, this document features methods for improving T cell therapy. The methods can include, or consist essentially of, administering, to a mammal and at a time that is from about 1 day before to about 360 days after the mammal receives an adoptive T cell therapy, a reversible BTK inhibitor that binds non-covalently to a BTK polypeptide, where T cells of the adoptive T cell therapy in the mammal express a reduced level of one or more polypeptides after the administering step as compared to the level of the one or more polypeptides expressed by T cells of an adoptive T cell therapy in a comparable mammal not administered the BTK inhibitor, where the one or more polypeptides are selected from the group consisting of an MIP-1α polypeptide, an MIP-1β polypeptide, a TNF-α polypeptide, a TNF-β polypeptide, an eotaxin polypeptide, an MCP-3 polypeptide, an IP-10 polypeptide, a VEGF polypeptide, an EGF polypeptide, a G-CSF polypeptide, an IFN-γ polypeptide, an FGF-2 polypeptide, a fractalkine polypeptide, an IL-1ra polypeptide, an IL-1β polypeptide, an IL-2 polypeptide, an IL-3 polypeptide, an IL-4 polypeptide, an IL-6 polypeptide, an IL-7 polypeptide, an IL-9 polypeptide, an IL-10 polypeptide, and an IL-17A polypeptide. The one or more polypeptides can include the MIP-1β polypeptide, the IP-10 polypeptide, and the TNF-α polypeptide. The one or more polypeptides can include the MIP-1β polypeptide, the TNF-α polypeptide, the IP-10 polypeptide, the IL-4 polypeptide, the IL-6 polypeptide, and the IL-10 polypeptide. The one or more polypeptides can include the MIP-1α polypeptide, the MIP-1β polypeptide, the eotaxin polypeptide, the MCP-3 polypeptide, the VEGF polypeptide, the IL-1ra polypeptide, the IL-4 polypeptide, and the IL-9 polypeptide. The one or more polypeptides can include the MIP-1α polypeptide, the MIP-1β polypeptide, the TNF-α polypeptide, the TNF-β polypeptide, the eotaxin polypeptide, the MCP-3 polypeptide, the IP-10 polypeptide, the VEGF polypeptide, the EGF polypeptide, the G-CSF polypeptide, the IFN-γ polypeptide, the FGF-2 polypeptide, the fractalkine polypeptide, the IL-1ra polypeptide, the IL-1β polypeptide, the IL-2 polypeptide, the IL-3 polypeptide, the IL-4 polypeptide, the IL-6 polypeptide, the IL-7 polypeptide, the IL-9 polypeptide, the IL-10 polypeptide, and the IL-17A polypeptide. The mammal can be a human. The mammal can have cancer, and the adoptive T cell therapy can treat the cancer following the administering step. The cancer can be lymphoma, leukemia, hepatocellular carcinoma, bowel cancer, lung cancer, breast cancer, ovarian cancer, melanoma, head and neck cancer, sarcoma, epithelial cancer, mesothelioma, thyroid cancer, brain cancer, or endocrine cancer. The mammal can have lymphoma, and the adoptive T cell therapy can treat the lymphoma following the administering step. The BTK inhibitor can be vecabrutinib, fenebrutinib, pirtobrutinib, ARQ531, XMU-MP3, CB1763, GNE-431, CGI-1746, RN1486, BMS-986142, HBW-3-10, or CG806. The T cells of the adoptive T cell therapy can include a chimeric antigen receptor. The chimeric antigen receptor can target a tumor-associated antigen. The tumor-associated antigen can be CD19. The adoptive T cell therapy can be a CAR T therapy. The adoptive T cell therapy can cause less expression of one or more cytokine polypeptides associated with cytokine release syndrome within the mammal than the level of expression of the one or more cytokine polypeptides within the comparable mammal that received the adoptive T cell therapy in the absence of the administration of the BTK inhibitor. The cytokine polypeptides can be an IL-6 polypeptide, an IL-10 polypeptide, an MIP-β polypeptide, an IP-10 polypeptide, or a TNF-α polypeptide. The BTK inhibitor can be administered to the mammal at a dose from about 10 mg/kg to about 250 mg/kg (e.g., from about 25 mg/kg to about 75 mg/kg). The BTK inhibitor can be administered to the mammal at least once a day. The BTK inhibitor can be administered to the mammal twice a day. The BTK inhibitor can be administered to the mammal within 1 to 2 months of the mammal receiving the T cell therapy.

In another aspect, this document features methods for improving T cell therapy. The methods can include, or consist essentially of, administering, to a mammal and at a time that is from about 1 day before to about 360 days after the mammal receives an adoptive T cell therapy, a reversible BTK inhibitor that binds non-covalently to a BTK polypeptide, where T cells of the adoptive T cell therapy in the mammal express an increased level of one or more polypeptides after the administering step as compared to the level of the one or more polypeptides expressed by T cells of an adoptive T cell therapy in a comparable mammal not administered the BTK inhibitor, where the one or more polypeptides are selected from the group consisting of an MCP-1 polypeptide and an IL-5 polypeptide. The one or more polypeptides can be the MCP-1 polypeptide. The mammal can be a human. The mammal can have cancer, and the adoptive T cell therapy can treat the cancer following the administering step. The cancer can be lymphoma, leukemia, hepatocellular carcinoma, bowel cancer, lung cancer, breast cancer, ovarian cancer, melanoma, head and neck cancer, sarcoma, epithelial cancer, mesothelioma, thyroid cancer, brain cancer, or endocrine cancer. The mammal can have lymphoma, and the adoptive T cell therapy can treat the lymphoma following the administering step. The BTK inhibitor can be vecabrutinib, fenebrutinib, pirtobrutinib, ARQ531, XMU-MP3, CB1763, GNE-431, CGI-1746, RN1486, BMS-986142, HBW-3-10, or CG806. The T cells of the adoptive T cell therapy can include a chimeric antigen receptor. The chimeric antigen receptor can target a tumor-associated antigen. The tumor-associated antigen can be CD19. The adoptive T cell therapy can be a CAR T therapy. The adoptive T cell therapy can cause less expression of one or more cytokine polypeptides associated with cytokine release syndrome within the mammal than the level of expression of the one or more cytokine polypeptides within the comparable mammal that received the adoptive T cell therapy in the absence of the administration of the BTK inhibitor. The cytokine polypeptides can be an IL-6 polypeptide, an IL-10 polypeptide, an MIP-β polypeptide, an IP-10 polypeptide, or a TNF-α polypeptide. The BTK inhibitor can be administered to the mammal at a dose from about 10 mg/kg to about 250 mg/kg (e.g., from about 25 mg/kg to about 75 mg/kg). The BTK inhibitor can be administered to the mammal at least once a day. The BTK inhibitor can be administered to the mammal twice a day. The BTK inhibitor can be administered to the mammal within 1 to 2 months of the mammal receiving the T cell therapy.

In another aspect, this document features methods for improving T cell therapy. The methods can include, or consist essentially of, administering, to a mammal and at a time that is from about 1 day before to about 360 days after the mammal receives an adoptive T cell therapy, a reversible BTK inhibitor that binds non-covalently to a BTK polypeptide, where T cells of the adoptive T cell therapy in the mammal have an increased percentage of target cell killing after the administering step as compared to the percentage of target cell killing by T cells of an adoptive T cell therapy in a comparable mammal not administered the BTK inhibitor. The percentage of target cell killing can be increased by at least 5 percent. The percentage of target cell killing can be increased by at least 10 percent. The percentage of target cell killing can be increased by at least 25 percent. The mammal can be a human. The mammal can have cancer, and the adoptive T cell therapy can treat the cancer following the administering step. The cancer can be lymphoma, leukemia, hepatocellular carcinoma, bowel cancer, lung cancer, breast cancer, ovarian cancer, melanoma, head and neck cancer, sarcoma, epithelial cancer, mesothelioma, thyroid cancer, brain cancer, or endocrine cancer. The mammal can have lymphoma, and the adoptive T cell therapy can treat the lymphoma following the administering step. The BTK inhibitor can be vecabrutinib, fenebrutinib, pirtobrutinib, ARQ531, XMU-MP3, CB1763, GNE-431, CGI-1746, RN1486, BMS-986142, HBW-3-10, or CG806. The T cells of the adoptive T cell therapy can include a chimeric antigen receptor. The chimeric antigen receptor can target a tumor-associated antigen. The tumor-associated antigen can be CD19. The adoptive T cell therapy can be a CAR T therapy. The adoptive T cell therapy can cause less expression of one or more cytokine polypeptides associated with cytokine release syndrome within the mammal than the level of expression of the one or more cytokine polypeptides within the comparable mammal that received the adoptive T cell therapy in the absence of the administration of the BTK inhibitor. The cytokine polypeptides can be an IL-6 polypeptide, an IL-10 polypeptide, an MIP-β polypeptide, an IP-10 polypeptide, or a TNF-α polypeptide. The BTK inhibitor can be administered to the mammal at a dose from about 10 mg/kg to about 250 mg/kg (e.g., from about 25 mg/kg to about 75 mg/kg). The BTK inhibitor can be administered to the mammal at least once a day. The BTK inhibitor can be administered to the mammal twice a day. The BTK inhibitor can be administered to the mammal within 1 to 2 months of the mammal receiving the T cell therapy.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIGS. 1A-1C. Reversible BTK inhibition with vecabrutinib in vitro enhances CART19 cell cytotoxicity and does not impair CART19 cells functions. FIG. 1A) Vecabrutinib increases CART19-mediated killing of tumor cells. CART19 and live JeKo-1 were co-cultured at 1:1 ratio for 24 hours in the presence of DMSO or vecabrutinib (1 μM or 10 μM). The percentage of killing were assessed relative to control (****p<0.0001, two-way ANOVA). FIG. 1B) Vecabrutinib increases CART19 proliferation at 10 μM, but has no statistically significant effect at 1 μM. CART19 and irradiated JeKo-1 were co-cultured at 1:1 ratio for 24 hours in the presence of DMSO or vecabrutinib (1 μM or 10 μM). Absolute CD3⁺ T cell counts were assessed by flow cytometry (**p<0.01, two-way ANOVA) . FIG. 1C) Vecabrutinib does not impair CART cell degranulation. JeKo-1 target cells were incubated with CART19 at a 5:1 ratio with monensin, hCD49d, and hCD28 in the presence of vecabrutinib at the indicated concentrations for 4 hours. Cells were then analyzed using intracellular flow cytometric assessment of CD107a as a marker for degranulation.

FIGS. 2A-2C. Vecabrutinib inhibits the secretion of inflammatory cytokines and chemokines in vitro. The supernatants that were harvested from FIG. 1A were analyzed by 38-cytokine multiplex (**p<0.01, ****p<0.0001; two-way ANOVA) . The levels of IL-6 (pg/mL) (FIG. 2A), IL-10 (pg/mL) (FIG. 2B), and MIP-1b (pg/mL) (FIG. 2C) are shown.

FIGS. 3A-3C. Vecabrutinib decreases the levels of multiple cytokines in patients with B cell malignancies four weeks after treatment with vecabrutinib. Sera of patients that maintained stable disease with vecabrutinib were obtained prior and 4 weeks post treatment with vecabrutinib and analyzed with 38-cytokine multiplex. The levels of IP-10 (FIG. 3A), MIP-1b (FIG. 3B), and TNF-α (FIG. 3C) are shown.

FIGS. 4A-4C. Reversible BTK inhibition in vivo enhances CAR T cell antitumor activity in xenograft models. FIG. 4A) The combination of vecabrutinib with CART19 cells resulted in synergistic anti-tumor effects in vivo. On day-14, NSG mice were injected with 1×10⁶ luciferase+JeKo-1 cells intravenously. Gavage began on day −1 with vecabrutinib 50 mg/kg BID or vehicle. Untransduced T cells (UTD) or CART19 were infused on day 0. Mice were imaged weekly to assess the tumor burden. p/s=photons/seconds. FIG. 4B) Vecabrutinib increases CART19 proliferation in vivo. Mice were bled weekly to measure CART19 by flow cytometry (*p<0.05, one-way ANOVA). FIG. 4C) Vecabrutinib decreases CRS. Mouse weight and wellbeing along with elevation of human cytokines represent a surrogate for the development of CRS intensity in mice receiving CART19. Mice were weighed weekly, and measurements are plotted as change in % weight from day 0 (**p<0.01, one-way ANOVA) .

FIGS. 5A-5F. Vecabrutinib induces transcriptomic changes that enhance proliferation of CART19 cells. Comparison of gene expression of CART19 cells that were co-cultured with irradiated JeKo-1 in the presence of vehicle (DMSO) or 1 μM or 10 μM vecabrutinib for 72 hours via RNA-Seq. Differential expression by heatmap (FIG. 5A for 1 μM Vec, and FIG. 5B for 10 μM Vec) or volcano plot (FIG. 5C for 1 μM Vec and FIG. 5D for 10 μM Vec) on RNA isolated from CART19 cells 72 hours after incubation with vehicle and vecabrutinib. FIG. 5E) Principal component analysis of overall gene expression patterns. FIG. 5F) Ingenuity pathway analysis predicts increased activation of the PI3K/AKT pathway in CART19 cells co-cultured with vecabrutinib (z-score 0.707, p=0.0012).

FIGS. 6A-6J. Plots of the normalized gene counts for significantly differentially expressed genes. CART19 cells were co-cultured with irradiated JeKo-1 in the presence of vehicle (DMSO) or 1 μM or 10 μM vecabrutinib for 72 hours via RNA-Seq. FIG. 6A) DOK-5: Downregulated. FIG. 6B) TRANK1: Upregulated. FIG. 6C) ADA2: Upregulated. FIG. 6D) CYSLTR1: Upregulated. FIG. 6E) LOC105373239: Upregulated. FIG. 6F) FHIT: Upregulated. FIG. 6G) KCNA6: Upregulated. FIG. 6H) P2RY10: Upregulated. FIG. 61) PDE3B: Upregulated. FIG. 6J) RESF1: Upregulated.

FIGS. 7A-7B. 10 μM vs Vehicle—GSEA Biological Process. FIG. 7A) Downregulated genes. FIG. 7B) Upregulated genes. CART19 cells that were co-cultured with irradiated JeKo-1 in the presence of vehicle (DMSO) or 10 μM vecabrutinib for 72 hours via RNA-Seq. Multiples pathways are enriched in CART19 cells that are co-cultured with 10 μM Vecabrutinib. Gene set enrichment analysis of significantly downregulated (FIG. 7A) and upregulated (FIG. 7B) genes using Enrichr (p-value<0.05).

FIG. 8. 1 μM vs Vehicle-GSEA Biological Process. Upregulated genes. CART19 cells that were co-cultured with irradiated JeKo-1 in the presence of vehicle (DMSO) or 1 μM vecabrutinib for 72 hours via RNA-Seq. Multiples pathways are enriched in CART19 cells that are co-cultured with 1 μM vecabrutinib. Gene set enrichment analysis of significantly upregulated genes using Enrichr (p-value<0.05).

FIG. 9. Canonical pathways from Ingenuity Pathway Analysis. CART19 cells that were co-cultured with irradiated JeKo-1 in the presence of vehicle (DMSO) or 10 μM vecabrutinib for 72 hours via RNA-Seq.

FIG. 10. CART19 cytotoxicity in the presence of Vecabrutinib versus the irreversible BTK inhibitor, Ibrutinib. CART19 or control cells (UTD) were cocultured at different effector-to-target ratio (E:T) with Jeko-1 cell line with vecabrutinib at 1 μM or 10 μM, or ibrutinib at 1 μM. At 24 hours, live Jeko cells numbers was measured by flow cytometry and percent killing was calculated relative to control (*p<0.05, **p<0.01, two-way ANOVA).

FIGS. 11A-11B. Proliferation. FIG. 11A) Vecabrutinib increases CART19 proliferation at 1 μM, but has no statistically significant effect at 10 μM, whereas the irreversible ibrutinib does not affect CART19 proliferation at 1 μM and significantly decreases it at 10 μM. CART19 and irradiated JeKo-1 were co-cultured at 1:1 ratio for 24 hours in the presence of DMSO or vecabrutinib (1 μM or 10 μM) or ibrutinib (1 μM or 10 μM). Absolute CD3⁺T cell counts were assessed by flow cytometry (*p<0.05, ****p<0.0001, two-way ANOVA). FIG. 11B) To further verify effect of BTK inhibition on CART19 proliferation, using different set of donors for CAR T cell production, CART19 and irradiated JeKo-1 were co-cultured at 1:1 ratio for 24 hours in the presence of DMSO or vecabrutinib (10 μM) or ibrutinib (10 μM). Vecabrutinib significantly increased CART19 proliferation at 10 μM, whereas ibrutinib decreases CART19 proliferation at 10 μM.

FIG. 12. Effect of BTK inhibitors on cytokine and chemokine secretions. CART19 and JeKo-1 were co-cultured at 1:1 ratio for 24 hours in the presence of DMSO (DMSO-JEKO1) or 1 μM vecabrutinib (1VEC-JEKO1) or 10 μM vecabrutinib (10VEC-JEKO1) , or 1 μM acalabrutinib (1ACA-JEKO1) or 10 acalabrutinib (10ACA-JEKO1) or 1 μM ibrutinib (1IBR-JEKO1). The supernatants were harvested and were analyzed by 38-cytokine multiplex ((*p<0.05, **p<0.01, ****p<0.0001; two-way ANOVA).

### DETAILED DESCRIPTION

This document provides methods and materials involved in improving adoptive T cell therapies (e.g., CAR T cell therapies). For example, one or more reversible BTK inhibitors that bind non-covalently to a BTK polypeptide (e.g. vecabrutinib, fenebrutinib, and/or pirtobrutinib) can be administered in combination with an adoptive cell therapy (e.g., a CART cell therapy) to improve one or more functions of a T cell. In some cases, a composition including one or more reversible BTK inhibitors that bind non-covalently to a BTK polypeptide (e.g. vecabrutinib, fenebrutinib, and/or pirtobrutinib) can be used to condition T cells (e.g., CAR T cells) ex vivo (e.g., for use in adoptive cell therapy). For example, a composition including one or more reversible BTK inhibitors that bind non-covalently to a BTK polypeptide (e.g. vecabrutinib, fenebrutinib, and/or pirtobrutinib) can be administered to T cells (e.g., CAR T cells) ex vivo to improve one or more functions of the T cells in adoptive T cell therapies (e.g., CAR T cell therapies). In some cases, a composition including one or more reversible BTK inhibitors that bind non-covalently to a BTK polypeptide (e.g. vecabrutinib, fenebrutinib, and/or pirtobrutinib) can be used to improve one or more functions of T cells (e.g., CAR T cells) in vivo. For example, a composition including one or more reversible BTK inhibitors that bind non-covalently to a BTK polypeptide (e.g. vecabrutinib, fenebrutinib, and/or pirtobrutinib) can be administered to a mammal being treated with an adoptive cell therapy (e.g., a CAR T cell therapy) to improve one or more functions of a T cell within the mammal (e.g., a T cell administered to the mammal during the adoptive cell therapy procedure). In some cases, one or more reversible BTK inhibitors that bind non-covalently to a BTK polypeptide (e.g., vecabrutinib, fenebrutinib, and/or pirtobrutinib) can be used in combination with an adoptive T cell therapy (e.g., a CAR T cell therapy) to reduce the number of cancer cells within a mammal.

As described herein, one or more reversible BTK inhibitors that bind non-covalently to a BTK polypeptide (e.g., vecabrutinib, fenebrutinib, and/or pirtobrutinib) used in combination with an adoptive T cell therapy (e.g., a CAR T cell therapy) can be used to improve one or more functions of a T cell (e.g., a CAR T cell). Examples of T cell functions of T cells of an adoptive T cell therapy (e.g., a CAR T cell therapy) that can be improved using one or more reversible BTK inhibitors that bind non-covalently to a BTK polypeptide (e.g., vecabrutinib, fenebrutinib, and/or pirtobrutinib) as described herein include, without limitation, T cell killing of target cells (e.g., targeted cancer cells), enhanced cytotoxicity, T cell proliferation, and cytokine and/or chemokine polypeptide release from T cells. For example, one or more reversible BTK inhibitors that bind non-covalently to a BTK polypeptide (e.g., vecabrutinib, fenebrutinib, and/or pirtobrutinib) can be used to increase the ability of T cells of an adoptive T cell therapy (e.g., a CAR T cell therapy) to kill target cells (e.g., targeted cancer cells). Also as described herein, one or more reversible BTK inhibitors that bind non-covalently to a BTK polypeptide (e.g., vecabrutinib, fenebrutinib, and/or pirtobrutinib) used in combination with an adoptive T cell therapy (e.g., a CAR T cell therapy) can be used with little or no effect on degranulation of a T cell (e.g., a CAR T cell).

In some cases, one or more reversible BTK inhibitors that bind non-covalently to a BTK polypeptide (e.g., vecabrutinib, fenebrutinib, and/or pirtobrutinib) can be used to alter the profile of cytokine and/or chemokine polypeptides released from T cells of an adoptive T cell therapy (e.g., a CAR T cell therapy). For example, one or more reversible BTK inhibitors that bind non-covalently to a BTK polypeptide (e.g., vecabrutinib, fenebrutinib, and/or pirtobrutinib) can be used to decrease the level of cytokine and/or chemokine polypeptides (e.g., MIP-1α polypeptides, MIP-1β polypeptides, eotaxin polypeptides, MCP-3 polypeptides, VEGF polypeptides, IL-1ra polypeptides, IL-4 polypeptides, and/or IL-9 polypeptides) released from T cells of an adoptive T cell therapy (e.g., a CAR T cell therapy). In some cases, one or more reversible BTK inhibitors that bind non-covalently to a BTK polypeptide (e.g., vecabrutinib, fenebrutinib, and/or pirtobrutinib) can be used to increase the level of cytokine and/or chemokine polypeptides (e.g., MCP-1 polypeptides) released from T cells of an adoptive T cell therapy (e.g., a CAR T cell therapy).

In some cases, one or more reversible BTK inhibitors that bind non-covalently to a BTK polypeptide (e.g., vecabrutinib, fenebrutinib, and/or pirtobrutinib) can be used to reduce the severity and/or likelihood of experiencing CRS, fevers, low blood pressure, neurotoxicity, and/or other side effects in a mammal treated with an adoptive T cell therapy (e.g., a CAR T cell therapy). For example, one or more reversible BTK inhibitors that bind non-covalently to a BTK polypeptide (e.g., vecabrutinib, fenebrutinib, and/or pirtobrutinib) can be used to reduce the likelihood that a mammal receiving an adoptive T cell therapy (e.g., a CAR T cell therapy) will experience CRS and/or neurotoxicity.

In some cases, one or more reversible BTK inhibitors that bind non-covalently to a BTK polypeptide (e.g., vecabrutinib, fenebrutinib, and/or pirtobrutinib) can be used to increase and/or maintain the proliferative potential of T cells of an adoptive T cell therapy (e.g., a CAR T cell therapy). For example, one or more reversible BTK inhibitors that bind non-covalently to a BTK polypeptide (e.g., vecabrutinib, fenebrutinib, and/or pirtobrutinib) can be used to increase the level of proliferation of T cells of an adoptive T cell therapy (e.g., a CAR T cell therapy). In some cases, one or more reversible BTK inhibitors that bind non-covalently to a BTK polypeptide (e.g., vecabrutinib, fenebrutinib, and/or pirtobrutinib) can be used to maintain the ability of T cells of an adoptive T cell therapy (e.g., a CAR T cell therapy) to proliferate after the adoptive T cell therapy is administered to the mammal.

In some cases, one or more reversible BTK inhibitors that bind non-covalently to a BTK polypeptide (e.g., vecabrutinib, fenebrutinib, and/or pirtobrutinib) can be used to increase and/or maintain the persistence of T cells of an adoptive T cell therapy (e.g., a CAR T cell therapy). For example, one or more reversible BTK inhibitors that bind non-covalently to a BTK polypeptide (e.g., vecabrutinib, fenebrutinib, and/or pirtobrutinib) can be used to increase the persistence of T cells of an adoptive T cell therapy (e.g., a CAR T cell therapy) after the adoptive T cell therapy is administered to the mammal. In some cases, one or more reversible BTK inhibitors that bind non-covalently to a BTK polypeptide (e.g., vecabrutinib, fenebrutinib, and/or pirtobrutinib) can be used to maintain the ability of T cells of an adoptive T cell therapy (e.g., a CAR T cell therapy) to persist for up to 36 months after the adoptive T cell therapy is administered to the mammal.

One or more reversible BTK inhibitors that bind non-covalently to a BTK polypeptide (e.g., vecabrutinib, fenebrutinib, and/or pirtobrutinib) can be used in combination with an adoptive T cell therapy (e.g., a CART cell therapy) to treat a mammal having any appropriate cancer. In some cases, a cancer treated as described herein can be a primary cancer. In some cases, a cancer treated as described herein can be a metastatic cancer. In some cases, a cancer can include one or more solid tumors. In some cases, a cancer can be a hematological cancer. In some cases, a cancer treated as described herein can be a refractory cancer. In some cases, a cancer treated as described herein can express a tumor-associated antigen (e.g., an antigenic substance produced by a cancer cell). Examples of cancers that can be treated as described herein include, without limitation, lymphomas (e.g., B cell lymphomas such as diffuse large cell lymphoma (DLBCL)), leukemias (e.g., chronic lymphocytic leukemia (CLL) and acute lymphoblastic leukemia (ALL)), germ cell tumors, hepatocellular carcinomas, bowel cancers, lung cancers, breast cancers, ovarian cancers, melanomas, head and neck cancers, sarcomas, epithelial cancers, mesothelioma, thyroid cancers, brain cancers, and endocrine cancers. For example, one or more reversible BTK inhibitors that bind non-covalently to a BTK polypeptide (e.g., vecabrutinib, fenebrutinib, and/or pirtobrutinib) can be used in combination with CAR T cell therapy to treat a lymphoma. In another example, one or more reversible BTK inhibitors that bind non-covalently to a BTK polypeptide (e.g., vecabrutinib, fenebrutinib, and/or pirtobrutinib) can be used in combination with CAR T cell therapy to treat a leukemia.

One or more reversible BTK inhibitors that bind non-covalently to a BTK polypeptide (e.g., vecabrutinib, fenebrutinib, and/or pirtobrutinib) can be used in combination with an adoptive T cell therapy (e.g., a CAR T cell therapy) targeting any appropriate antigen within a mammal (e.g., a mammal having cancer). In some cases, an antigen can be a tumor-associated antigen (e.g., an antigenic substance produced by a cancer cell). Examples of tumor associated antigens that can be targeted by an adoptive T cell therapy provided herein include, without limitation, cluster of differentiation 19 (CD19; associated with B cell lymphomas, acute lymphoblastic leukemia (ALL), and chronic lymphocytic leukemia (CLL)), alphafetoprotein (AFP; associated with germ cell tumors and/or hepatocellular carcinoma), carcinoembryonic antigen (CEA; associated with bowel cancer, lung cancer, and/or breast cancer), CA-125 (associated with ovarian cancer), mucin 1 (MUC-1; associated with breast cancer), epithelial tumor antigen (ETA; associated with breast cancer), melanoma-associated antigen (MAGE; associated with malignant melanoma), CD22 (associated with B cell malignancies), CD20 (associated with B cell malignancies), kappa or lambda (associated with B cell malignancies), BCMA (associated with multiple myeloma), CS1 (associated with multiple myeloma), CD123 (associated with myeloid malignancies), CD33 (associated with myeloid malignancies), CLL-1 (associated with myeloid malignancies), FLT3 (associated with myeloid malignancies), CD30 (associated with Hodgkin lymphoma), CD5 (associated with T cell malignancies), CD2 (associated with T cell malignancies), CD7 (associated with T cell malignancies), CD123 (associated with Hodgkin lymphoma), TSHR (associated with thyroid cancer), mesothelin (associated with mesothelioma), PSMA (associated with prostate cancer), ICAM (associated with thyroid cancer), EGFR (associated with skin cancer, colon cancer, and epithelial cancers), EGFRviii (associated with brain cancer), and IL13R2 (b associated with rain cancer). For example, one or more reversible BTK inhibitors that bind non-covalently to a BTK polypeptide (e.g., vecabrutinib, fenebrutinib, and/or pirtobrutinib) can be used in combination with a CAR T cell therapy targeting CD19 (e.g., CART19 cell therapy) to treat cancer as described herein.

Any type of mammal can be treated as described herein. Examples of mammals that can be treated as described herein include, without limitation, primates (e.g., humans and monkeys), dogs, cats, horses, cows, pigs, sheep, rabbits, mice, and rats. In some cases, a mammal can be identified as having cancer or another condition in need of adoptive T cell therapy (e.g., CAR T cell therapy). Any appropriate method can be used to identify a mammal as having cancer. Once identified as having cancer, the mammal can be administered, or instructed to self-administer, one or more reversible BTK inhibitors that bind non-covalently to a BTK polypeptide (e.g., vecabrutinib, fenebrutinib, and/or pirtobrutinib) as described herein.

Any appropriate reversible BTK inhibitor that binds non-covalently to a BTK polypeptide can be used as described herein. Examples of reversible BTK inhibitors that bind non-covalently to a BTK polypeptide and can be used as described herein include, without limitation, vecabrutinib (CAS No.: 1510829-06-7), fenebrutinib (CAS No.: 1434048-34-6), pirtobrutinib (CAS No.: 2101700-15-4), ARQ531 (CAS No.: 2095393-15-8), XMU-MP 3 (CAS No.: 2031152-08-4), CB 1763, GNE-431 (CAS No. 1433820-83-7), CGI-1746, RN1486, BMS-986142, HBW-3-10, CG806, and GT-1530. An example of a BTK polypeptide includes, without limitation, the BTK polypeptides having the amino acid sequence set forth in National Center for Biotechnology Information (NCBI) accession no. NP_000052.

In some cases, one or more (e.g., one, two, three, four, five, or more) reversible BTK inhibitors that bind non-covalently to a BTK polypeptide can be administered in combination with an adoptive T cell therapy (e.g., a CAR T cell therapy) to a mammal (e.g., a mammal having cancer) to improve one or more functions of a T cell of the adoptive T cell therapy within the mammal. For example, two or more reversible BTK inhibitors that bind non-covalently to a BTK polypeptide (e.g., vecabrutinib and fenebrutinib) can be administered to a mammal having cancer (e.g., a human having cancer) and treated with an adoptive T cell therapy (e.g., a CAR T cell therapy) to improve one or more functions of a T cell (e.g., a CAR T cell) within the mammal.

One or more reversible BTK inhibitors that bind non-covalently to a BTK polypeptide (e.g. vecabrutinib, fenebrutinib, and/or pirtobrutinib) used in combination with an adoptive T cell therapy (e.g., a CAR T cell therapy) can be administered in any appropriate order with respect to the administration of the adoptive T cell therapy. In some cases, a composition including one or more reversible BTK inhibitors that bind non-covalently to a BTK polypeptide (e.g. vecabrutinib, fenebrutinib, and/or pirtobrutinib) can be administered concurrently with the administration of the adoptive T cell therapy. For example, in cases where one or more reversible BTK inhibitors that bind non-covalently to a BTK polypeptide (e.g. vecabrutinib, fenebrutinib, and/or pirtobrutinib) are administered to a mammal (e.g., in vivo administration), a composition including one or more reversible BTK inhibitors that bind non-covalently to a BTK polypeptide (e.g. vecabrutinib, fenebrutinib, and/or pirtobrutinib) can be administered to the mammal any time during the course of an adoptive T cell therapy procedure. In some cases, a composition including one or more reversible BTK inhibitors that bind non-covalently to a BTK polypeptide (e.g. vecabrutinib, fenebrutinib, and/or pirtobrutinib) can be administered in series with the administration of the adoptive T cell therapy. For example, in cases where one or more reversible BTK inhibitors that bind non-covalently to a BTK polypeptide (e.g. vecabrutinib, fenebrutinib, and/or pirtobrutinib) are administered to a mammal (e.g., in vivo administration), a composition including one or more reversible BTK inhibitors that bind non-covalently to a BTK polypeptide (e.g. vecabrutinib, fenebrutinib, and/or pirtobrutinib) can be administered before, during, after, both before and after, or both during and after the adoptive T cell therapy procedure. In some cases, a composition including one or more reversible BTK inhibitors that bind non-covalently to a BTK polypeptide (e.g. vecabrutinib, fenebrutinib, and/or pirtobrutinib) can be administered before, during, and after the adoptive T cell therapy procedure. For example, a composition including one or more reversible BTK inhibitors that bind non-covalently to a BTK polypeptide (e.g. vecabrutinib, fenebrutinib, and/or pirtobrutinib) can be administered to a mammal (e.g., a human) from about 1 day before to about 360 days after the mammal has been received an adoptive T cell therapy procedure. For example, a composition including one or more reversible BTK inhibitors that bind non-covalently to a BTK polypeptide (e.g. vecabrutinib, fenebrutinib, and/or pirtobrutinib) can be administered to a mammal (e.g., a human) within 1 to 2 months of said mammal receiving said T cell therapy.

In some cases, one or more reversible BTK inhibitors that bind non-covalently to a BTK polypeptide (e.g. vecabrutinib, fenebrutinib, and/or pirtobrutinib) can be used ex vivo. For example, a composition including one or more reversible BTK inhibitors that bind non-covalently to a BTK polypeptide (e.g. vecabrutinib, fenebrutinib, and/or pirtobrutinib) can be administered to T cells (e.g., CAR T cells) of an adoptive T cell therapy procedure ex vivo. In some cases, T cells of an adoptive T cell therapy procedure can be contacted with a composition including one or more reversible BTK inhibitors that bind non-covalently to a BTK polypeptide (e.g. vecabrutinib, fenebrutinib, and/or pirtobrutinib) at the time the T cells are being administered nucleic acid encoding a CAR. For example, T cells can be exposed to nucleic acid encoding a CAR and a composition including one or more reversible BTK inhibitors that bind non-covalently to a BTK polypeptide (e.g. vecabrutinib, fenebrutinib, and/or pirtobrutinib) to produce T cells for an adoptive T cell therapy procedure.

One or more reversible BTK inhibitors that bind non-covalently to a BTK polypeptide (e.g. vecabrutinib, fenebrutinib, and/or pirtobrutinib) used in combination with an adoptive T cell therapy (e.g., a CART cell therapy) can be administered to a mammal having a cancer as a combination therapy with one or more additional agents used to treat a cancer. For example, one or more reversible BTK inhibitors that bind non-covalently to a BTK polypeptide (e.g. vecabrutinib, fenebrutinib, and/or pirtobrutinib) used in combination with an adoptive cell therapy can be administered to a mammal in combination with one or more anti-cancer treatments (e.g., radiation therapy, chemotherapy, targeted therapies, hormonal therapy, angiogenesis inhibitors, checkpoint inhibitors, oncolytic virus therapy, T cell immunotherapy, and/or radiofrequency ablation). In cases where a composition including one or more reversible BTK inhibitors that bind non-covalently to a BTK polypeptide (e.g. vecabrutinib, fenebrutinib, and/or pirtobrutinib) used in combination with an adoptive cell therapy is used with additional agents treat a cancer, the one or more additional agents can be administered at the same time or independently. In some cases, a composition including one or more reversible BTK inhibitors that bind non-covalently to a BTK polypeptide (e.g. vecabrutinib, fenebrutinib, and/or pirtobrutinib) used in combination with an adoptive cell therapy can be administered first, and the one or more additional agents administered second, or vice versa.

In some cases, one or more reversible BTK inhibitors that bind non-covalently to a BTK polypeptide (e.g. vecabrutinib, fenebrutinib, and/or pirtobrutinib) used in combination with an adoptive T cell therapy (e.g., a CART cell therapy) can be formulated into a pharmaceutically acceptable composition for administration to a mammal having cancer. For example, a therapeutically effective amount of a reversible BTK inhibitor that binds non-covalently to a BTK polypeptide (e.g. vecabrutinib, fenebrutinib, or pirtobrutinib) can be formulated together with one or more pharmaceutically acceptable carriers (additives) and/or diluents. A pharmaceutical composition can be formulated for administration in solid or liquid form including, without limitation, sterile solutions, suspensions, sustained-release formulations, tablets, capsules, pills, powders, and granules. Pharmaceutically acceptable carriers, fillers, and vehicles that may be used in a pharmaceutical composition described herein include, without limitation, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol such as Vitamin E TPGS, sodium carboxymethylcellulose, polyacrylates, waxes, polyethylene-polyoxypropylene-block polymers, and wool fat.

A composition (e.g., a pharmaceutical composition) containing one or more reversible BTK inhibitors that bind non-covalently to a BTK polypeptide (e.g. vecabrutinib, fenebrutinib, and/or pirtobrutinib) used in combination with an adoptive T cell therapy (e.g., a CART cell therapy) can be designed for oral or parenteral (including subcutaneous, intramuscular, intravenous, intratumoral, and intradermal) administration. When being administered orally, a pharmaceutical composition containing one or more reversible BTK inhibitors that bind non-covalently to a BTK polypeptide (e.g. vecabrutinib, fenebrutinib, and/or pirtobrutinib) can be in the form of a pill, tablet, or capsule. Compositions suitable for parenteral administration include aqueous and non-aqueous sterile injection solutions that can contain anti-oxidants, buffers, bacteriostats, and solutes which render the formulation isotonic with the blood of the intended recipient; and aqueous and non-aqueous sterile suspensions which may include suspending agents and thickening agents. The formulations can be presented in unit-dose or multi-dose containers, for example, sealed ampules and vials, and may be stored in a freeze dried (lyophilized) condition requiring only the addition of the sterile liquid carrier, for example water for injections, immediately prior to use. Extemporaneous injection solutions and suspensions may be prepared from sterile powders, granules, and tablets.

A composition (e.g., a pharmaceutical composition) containing one or more reversible BTK inhibitors that bind non-covalently to a BTK polypeptide (e.g. vecabrutinib, fenebrutinib, and/or pirtobrutinib) used in combination with an adoptive T cell therapy (e.g., a CART cell therapy) can be administered locally or systemically. For example, a composition containing one or more reversible BTK inhibitors that bind non-covalently to a BTK polypeptide (e.g. vecabrutinib, fenebrutinib, and/or pirtobrutinib) can be administered systemically by an oral administration or by injection to a mammal (e.g., a human).

Effective doses of one or more reversible BTK inhibitors that bind non-covalently to a BTK polypeptide (e.g. vecabrutinib, fenebrutinib, or pirtobrutinib) can vary depending on the severity of the condition being treated (e.g., the severity of a cancer being treated), the route of administration, the age and general health condition of the subject, excipient usage, the possibility of co-usage with other therapeutic treatments such as use of other agents, and the judgment of the treating physician.

An effective amount of a composition containing one or more reversible BTK inhibitors that bind non-covalently to a BTK polypeptide (e.g. vecabrutinib, fenebrutinib, or pirtobrutinib) can be any amount that improves one or more functions of a T cell of an adoptive T cell therapy (e.g., a CAR T cell therapy) without producing significant toxicity to the mammal. An effective amount of a reversible BTK inhibitor that binds non-covalently to a BTK polypeptide such as vecabrutinib, fenebrutinib, or pirtobrutinib can be from about 10 mg/kg (e.g., 10 mg/kg BID) to about 250 mg/kg (e.g., 250 mg/kg BID).

In some cases, an effective amount of vecabrutinib, fenebrutinib, or pirtobrutinib can be from about 10 mg/kg to about 250 mg/kg, from about 10 mg/kg to about 200 mg/kg, from about 10 mg/kg to about 150 mg/kg, from about 10 mg/kg to about 100 mg/kg, from about 10 mg/kg to about 75 mg/kg, from about 10 mg/kg to about 50 mg/kg, from about 10 mg/kg to about 25 mg/kg, from about 25 mg/kg to about 250 mg/kg, from about 50 mg/kg to about 250 mg/kg, from about 75 mg/kg to about 250 mg/kg, from about 100 mg/kg to about 250 mg/kg, from about 150 mg/kg to about 250 mg/kg, from about 200 mg/kg to about 250 mg/kg, from about 50 mg/kg to about 200 mg/kg, from about 100 mg/kg to about 150 mg/kg, from about 50 mg/kg to about 100 mg/kg, or from about 150 mg/kg to about 200 mg/kg. For example, an effective amount of vecabrutinib, fenebrutinib, or pirtobrutinib can be about 50 mg/kg. The effective amount can remain constant or can be adjusted as a sliding scale or variable dose depending on the mammal's response to treatment. Various factors can influence the actual effective amount used for a particular application. For example, the frequency of administration, duration of treatment, use of multiple treatment agents, route of administration, and severity of the condition (e.g., a cancer) may require an increase or decrease in the actual effective amount administered. For ex vivo use, an effective amount of a reversible BTK inhibitor that binds non-covalently to a BTK polypeptide such as vecabrutinib, fenebrutinib, or pirtobrutinib can be from about 1 μM to about 80 μM (e.g., from about 1 μM to about 60 μM, from about 1 μM to about 50 μM, from about 1 μM to about 40 μM, from about 1 μM to about 30 μM, from about 1 μM to about 20 μM, from about 1 μM to about 10 μM, from about 10 μM to about 100 μM, from about 20 μM to about 100 μM, from about 30 μM to about 100 μM, from about 40 μM to about 100μM, from about 50 μM to about 100 μM, from about 60 μM to about 100 μM, from about 70 μM to about 100 μM, from about 80 μM to about 100 μM, from about 10 μM to about 80 μM, from about 20 μM to about 60 μM, from about 30 μM to about 50 μM, from about 10 μM to about 20μM, from about 20 μM to about 40 μM, from about 40 μM to about 60 μM, from about 50 μM to about 70 μM, or from about 60 μM to about 80 μM). For example, for ex vivo use an effective amount of vecabrutinib, fenebrutinib, or pirtobrutinib can be about 10 μM.

The frequency of administration of a composition containing one or more reversible BTK inhibitors that bind non-covalently to a BTK polypeptide (e.g. vecabrutinib, fenebrutinib, and/or pirtobrutinib) can be any frequency that improves one or more functions of a T cell of an adoptive T cell therapy (e.g., a CAR T cell therapy) without producing significant toxicity to the mammal. For example, the frequency of administration can be from about once a week to about three times a day, from about twice a month to about six times a day, or from about twice a week to about once a day. In some cases, the frequency of administration can be from once a day to four times a day. The frequency of administration can remain constant or can be variable during the duration of treatment. A course of treatment with a composition containing one or more reversible BTK inhibitors that bind non-covalently to a BTK polypeptide (e.g. vecabrutinib, fenebrutinib, and/or pirtobrutinib) can include rest periods. For example, a composition containing one or more reversible BTK inhibitors that bind non-covalently to a BTK polypeptide (e.g. vecabrutinib, fenebrutinib, and/or pirtobrutinib) can be administered daily over a two-week period followed by a two-week rest period, and such a regimen can be repeated multiple times. As with the effective amount, various factors can influence the actual frequency of administration used for a particular application. For example, the effective amount, duration of treatment, use of multiple treatment agents, route of administration, and severity of the condition (e.g., a cancer) may require an increase or decrease in administration frequency.

An effective duration for administering a composition containing one or more reversible BTK inhibitors that bind non-covalently to a BTK polypeptide (e.g. vecabrutinib, fenebrutinib, and/or pirtobrutinib) can be any duration that improves one or more functions of a T cell of an adoptive T cell therapy (e.g., a CAR T cell therapy) without producing significant toxicity to the mammal. For example, the effective duration can vary from several days to several weeks, months, or years. In some cases, the effective duration for the treatment of a cancer can range in duration from about one month to about 10 years. Multiple factors can influence the actual effective duration used for a particular treatment. For example, an effective duration can vary with the frequency of administration, effective amount, use of multiple treatment agents, route of administration, and severity of the condition being treated.

In some cases, one or more functions of T cells (e.g., CAR T cells) can be monitored. Any appropriate method can be used to determine whether or not one or more functions of T cells (e.g., T cells of an adoptive T cell therapy such as a CAR T cell) are improved at different time points. Examples of methods that can be used to evaluate T cell (e.g., CAR T cell) functions include, without limitation, cytotoxicity assays (e.g., to evaluate whether or not T cells (e.g., CAR T cells) are effective at killing target cells), cytokine assays (e.g., to evaluate whether or not cytokine and/or chemokine polypeptides related to CRS are being produced), exhaustion assays (e.g., to evaluate whether or not expression of inhibitory molecules is suppressed), cell number determinations, and proliferation assays.

In some cases, the number of cancer cells present within a mammal, and/or the severity of one or more symptoms related to a cancer being treated can be monitored. Any appropriate method can be used to determine whether or not the number of cancer cells present within a mammal is reduced. For example, imaging techniques can be used to assess the number of cancer cells present within a mammal.

A composition containing one or more reversible BTK inhibitors that bind non-covalently to a BTK polypeptide (e.g. vecabrutinib, fenebrutinib, and/or pirtobrutinib) can be combined with packaging material and configured into a kit. The packaging material included in a kit can contain instructions or a label describing how the composition can be used, for example, in combination with an adoptive cell therapy (e.g., CAR T cell therapy) to improve one or more functions of a T cell (e.g., a CAR T cell) of an adoptive T cell therapy as described herein. In some cases, a kit also can include materials for use in an adoptive cell therapy (e.g., CAR T cell therapy) procedure.

The invention will be further described in the following examples, which do not limit the scope of the invention described in the claims.

### EXAMPLES

### Example 1: Enhanced Car T Cell Activity with Non-covalent BTK Inhibition

This Example demonstrates that BTK inhibition using a BTK inhibitor that inhibits BTK polypeptide activity via non-covalent binding such as vecabrutinib can improve CART19 antitumor efficacy through the reversible inhibition of BTK.

### MATERIALS AND METHODS

### Cell Lines and Clinical Samples

The mantle cell lymphoma cell line Jeko-1 was obtained from ATCC (Manassas, VA, USA). This cell line was transduced with a with a firefly luciferase ZsGreen (Addgene, Cambridge, MA, USA), and then sorted to obtain >99% positive population that was maintained in either R10 or R20 (RPMI 1640, Gibco, Gaithersburg, MD, US) media containing 10% or 20% fetal bovine serum (FBS, Millipore Sigma, Ontario, Canada), and 1% penicillin-streptomycin-glutamine (Gibco, Gaithersburg, MD, US). Cell lines were kept in culture up to 20 passages, and fresh aliquots were thawed every 7-8 weeks. Clinically annotated serum products of patients with different B cell malignancies from the phase-1 clinic trail (NCT03037645) were obtained from Sunesis (San Francisco, CA).

### Generation of Cart19 Cells

Peripheral blood mononuclear cells (PBMC) were isolated from de-identified normal donor blood apheresis cones using SepMate tubes (STEMCELL Technologies, Vancouver, Canada). T cells were separated with negative selection magnetic beads using EasySep™ Human T Cell Isolation Kit (STEMCELL Technologies, Vancouver, Canada). Primary cells were cultured in T Cell Medium made with X-Vivo 15 (Lonza, Walkersville, MD, USA) supplemented with 10% human serum albumin (Corning, NY, USA) and 1% penicillin-streptomycin-glutamine (Gibco, Gaithersburg, MD, USA). CART19 cells were generated through the lentiviral transduction of normal donor T cells. A second generation 4-1BB costimulated CAR construct (FMC63-41BBz) was synthesized. Lentiviral particles were generated through the transient transfection of plasmid into 293T virus producing cells in the presence of Lipofectamine 3000 (Invitrogen, Carlsbad, CA, USA), VSV-G, and packaging plasmids (Addgene, Cambridge, MA, USA). T cells isolated from normal donors were stimulated using Cell Therapy Systems Dynabeads CD3/CD28 (Life Technologies, Oslo, Norway) at a 1:3 ratio and then transduced with lentivirus particles 24 hours after stimulation at a multiplicity of infection (MOI) of 3.0. CAR T cells were maintained in TCM for 5 days. At day 6, magnetic removal was performed on CAR T cells, and they were cryopreserved on day 8 in liquid nitrogen for future experiments.

### T Cell Functional Assays

For proliferation assays, CART19 cells were cultured with irradiated CD19⁺ cell line Jeko-1 at a 1:1 ratio for 24 hours. Then, the cells were harvested and washed with flow buffer, following by surface staining with anti-hCD3 (eBioscience, San Diego, CA, USA) and LIVE/DEAD™ Fixable Aqua Dead Cell Stain Kit (Invitrogen, Carlsbad, CA, USA). Cells were washed twice with BD stain buffer (BD Horizon, Franklin Lakes, NJ). Cytometric data were acquired using a CytoFLEX Flow Cytometer (Beckman Coulter, Chaska, MN, USA). Gating was performed using FlowJo X10.0.7r2 software (Ashland, OR, USA). Absolute quantification was obtained using volumetric measurement or CountBright absolute counting beads (Invitrogen, Carlsbad, CA, USA). For Degranulation assays, T cells treated with DMSO or vecabrutinib were incubated with target cells at an effector: target ratio of 1:5. Antibodies against FITC-CD107a (BD Pharmingen, San Diego, CA, USA, Cat #555800), CD28 (BD Biosciences, San Diego, CA, USA, Cat #348040), CD49d (BD Biosciences, San Diego, CA, USA, Cat #340976), and monensin (Biolegend, San Diego, CA, USA, Cat #420701) were added prior to the incubation. After 4 hours, cells were harvested and stained with LIVE/DEAD™ Fixable Aqua. Cells were then fixed and permeabilized (FIX & PERM Cell Fixation & Cell Permeabilization Kit, Life Technologies, Oslo, Norway, Cat #GAS004) and stained for CD3 (clone UCHT1) APC (Cat #17-0038-42, eBioscience, San Diego, CA, USA).

### Cytokine Measurement

Cytokine analysis was performed on 72-hour cell supernatant obtained from the proliferation assays. Debris was removed from the supernatant by centrifugation at 10,000× g for 5 minutes. Supernatant was then diluted 1:2 with assay buffer before following the manufacturer's protocol for Milliplex Human Cytokine/Chemokine MAGNETIC BEAD Premixed 38 Plex Kit (HCYTMAG-60K-PX38, Millipore Sigma, Ontario, Canada). Data were collected using Luminex (Millipore Sigma, Ontario, Canada). The same procedure was also performed on serum samples obtained from Sunesis (San Francisco, CA).

### In Vivo Studies

6-8 week old, non-obese diabetic/severe combined immunodeficient female mice bearing a targeted mutation in the interleukin (IL)-2 receptor gamma chain gene (NSG) were obtained from Jackson Laboratories (Jackson Laboratories, Bar Harbor, ME, USA). Mice were maintained in an animal barrier space. Mice were intravenously injected with 1.0×10⁶ luciferase+JeKo-1 cells. Fourteen days after injection, mice were imaged with a bioluminescent imager using an IVIS® Lumina S5 Imaging System (PerkinElmer, Hopkinton, MA, USA) to confirm engraftment. Imaging was performed 10 minutes after the intraperitoneal injection of 10 μL/g D-luciferin (15 mg/mL, Gold Biotechnology, St. Louis, MO, USA). Mice were then randomized based on their bioluminescence imaging to receive either untransduced T cells (UTD) or CART19 cells. CART19 mice were then randomized to receive vehicle or vecabrutinib via oral gavage. UTD mice received vehicle only. Mice were weighed weekly, and measurements were recorded in grams. Tumor burden was measured weakly by imaging mice with a bioluminescent imager using an IVIS® Lumina S5 Imaging System (PerkinElmer, Hopkinton, MA, USA), and measurements were recorded in total flux (p/s). Tail vein bleeding was done 7-8 days after injection of CAR T cells to assess T cell expansion (by measuring CD3 cells using flow cytometry) and cytokines and chemokines, and weekly thereafter. Briefly, mouse peripheral blood was lysed using BD FACS Lyse buffer (BD Biosciences, San Jose, CA, USA) and stained with anti-human CD3 PE-Cy7 (BioLegend, San Diego, CA, USA), anti-human CD45 BV421, anti-human CD20 PE, anti-mouse CD45 (clone 30-F11) APC-Cy 7 (BioLegend, San Diego, CA, USA), and LIVE/DEAD™ Fixable Aqua Dead Cell Stain Kit (Invitrogen, Carlsbad, CA, USA). Cells were washed twice with BD stain buffer (BD Horizon, Franklin Lakes, NJ). Cytometric data were acquired using a CytoFLEX Flow Cytometer (Beckman Coulter, Chaska, MN, USA). Gating was performed using FlowJo X10.0.7r2 software (Ashland, OR, USA). Absolute quantification was obtained using volumetric measurement or CountBright absolute counting beads (Invitrogen, Carlsbad, CA, USA). Mice were euthanized for necropsy when moribund.

### Vecabrutinib Preparation

Vecabrutinib was obtained from Sunesis in a powder form at room temperature. Vecabrutinib solution was prepared once weekly and stored at 4° C. The desired amount of vecabrutinib was weighed and placed in a 15 ml conical tube. Vehicle solution (0.5% CMC/0.1% Tween 80 in water) was then added. The solution was then vortexed to suspend the drug in vehicle to the extent that no material is settled on the bottom. The solution was then sonicated in a water bath for around 30 minutes, with intermittent vortexing ensuring material is suspended in vehicle and not clinching to the vessel. Vecabrutinib was administered to the mice via oral gavage at a dose of 50 mg/kg twice daily for 4 weeks excluding weekends.

### RNA Isolation and RNA-Seq on the Supernatant of Stimulated CART19 Cells

CART19 and irradiated Jeko-1 cells were co-cultured at a 1:1 ration for 72 hours with DMSO or 10 μM Vecabrutinib. Three biological replicates of stimulated and unstimulated CART19 cells were included.

RNA was isolated using miRNeasy Micro kit (Qiagen, Gaithersburg, MD, USA) and treated with RNase-Free DNase Set (Qiagen, Gaithersburg, MD, USA). Total RNA was prepped with a SMARTer stranded total RNA-seq kit v2, Pico input mammalian (Takara, Mountain View, CA, USA). Total RNA (three samples per lane) was sequenced on an Illumina HiSeq 4000 (Illumina, San Diego, CA, USA). Library preparation and sequencing were performed by the Medical Genome Facility Genome, Analysis Core (Mayo Clinic, Rochester, MN, USA). Fastq files were viewed in FastQC v0.11.8 to check for quality. Adaptor sequences were removed using Cutadapt v1.18.46. Output files were re-checked for quality and adaptor removal using FastQC v0.11.8. The latest human (GRCh38) reference genomes were downloaded from NCBI. Genome index files were generated using STAR, and the paired end reads were mapped to the genome for each condition. HTSeq was used to generate expression counts for each gene, and DeSeq2 was used to calculate differential expression using adjusted p values <0.05. Heatmap was created using pheatmap (cran.r-project.org/web/packages/pheatmap/index.html). Networks were generated using Ingenuity Pathway Analysis v49932394 (QIAGEN, qiagenbioinformatics.com/products/ingenuity-pathway-analysis). Gene set enrichment analyses were performed using Enrichr (maayanlab.cloud/Enrichr/).

### RESULTS

### Reversible BTK inhibition with Vecabrutinib in Vitro Enhances CART19 Cell Cytotoxicity and Does Not Impair CART19 Cells Proliferation and Degranulation

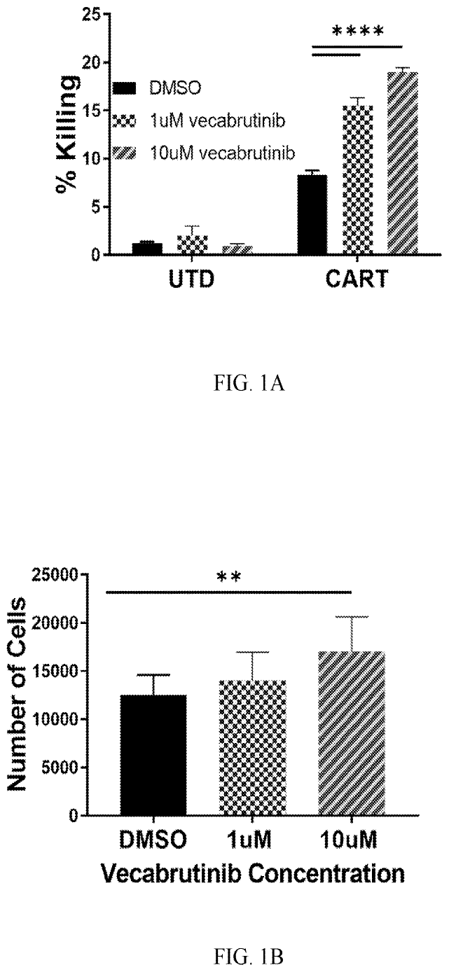

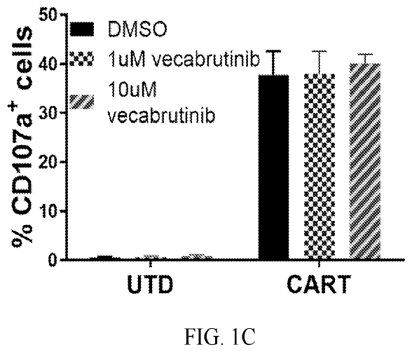

To investigate CAR T-specific cytotoxicity, CART19 cells or control UTD T cells were co-cultured with the CD19⁺ Mantle cell lymphoma cell line, Jeko-1, at 1:1 ratio and treated with vehicle (DMSO) or vecabrutinib at 1 μM or 10 μM for 24 hours (FIG. 1A). There was a dose-dependent increase in tumor specific cytotoxicity in the presence of vecabrutinib. Vecabrutinib did not exhibit independent tumor activity (FIG. 1A), indicating that the enhanced cytotoxicity is due to a direct effect on CART19 cells.

Next, the effect of vecabrutinib on CART19 proliferation was assessed. CART19 cells were cocultured with irradiated JeKo-1 cells for 24 hours in the presence of vecabrutinib (1 μM or 10 μM), and the absolute count of CART19 was measured by flow cytometry and used as a surrogate for proliferation. When used at 1 μM, vecabrutinib had no significant effect on CAR T cell antigen specific proliferation; however, there was a significant increase in CAR T cell proliferation when incubated with the higher dose of vecabrutinib (10 μM) (FIG. 1B). To further validate that vecabrutinib does not impair effector CAR T cell function, CART19 cells incubated with Jeko-1 in the presence of increasing concentrations of vecabrutinib showed no difference in degranulation as compared to cells incubated with vehicle control (FIG. 1C). The higher the percentage of CD107a cells means that these cells are active and degranulated. Overall, these results indicate that vecabrutinib enhances CART19 cell mediated cytotoxicity, without affecting their proliferative potential, demonstrating that reversible BTK inhibition improves CART cell-mediated efficacy.

### Vecabrutinib Decreases the Levels of Multiple Inflammatory Cytokines in Vitro

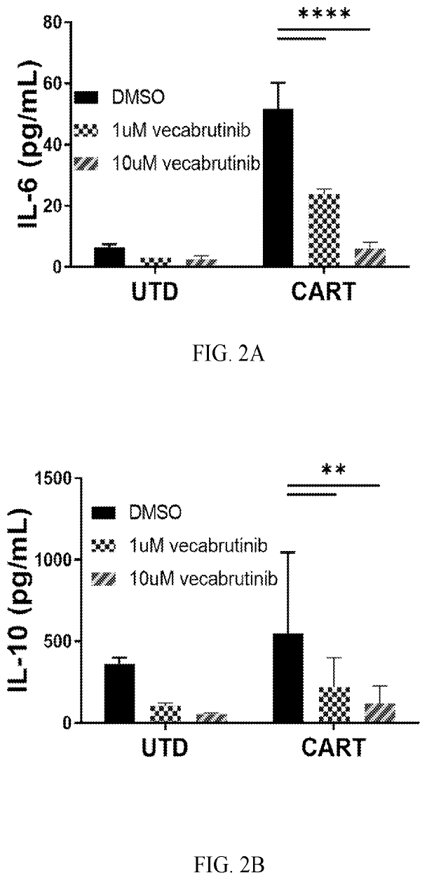

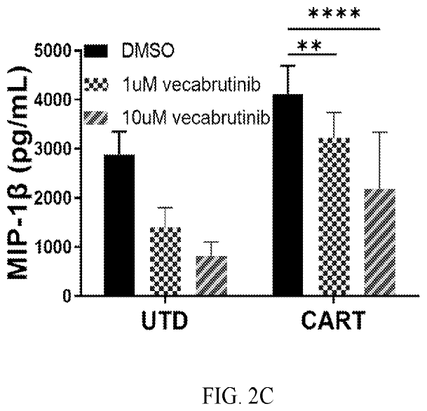

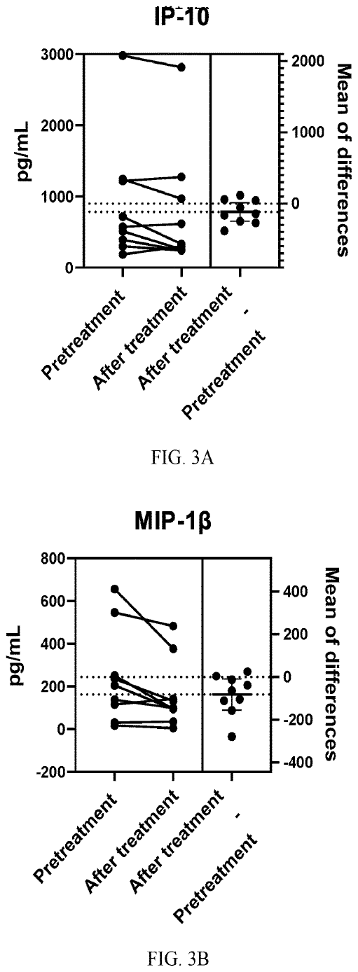

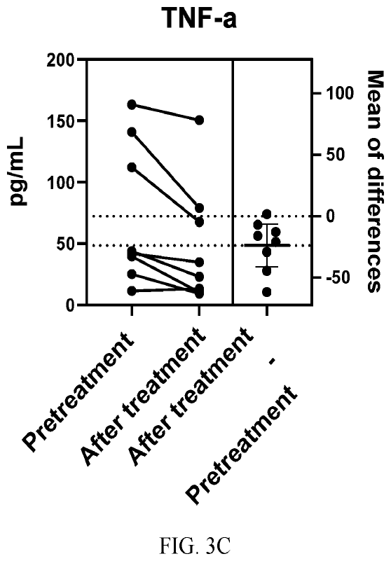

The effect of vecabrutinib on cytokine production was determined. The levels of multiple pro-inflammatory cytokines were measured in the supernatant of CART19 cells co-cultured for 3 days with JeKo-1 and increasing concentration of vecabrutinib (1 μM and 10 μM). There was a significant and dose dependent decrease in multiple cytokines that are known to be involved in the development of CRS such IL-6, IL-10, and MIP-1B (FIGS. 2A-2C). The serum cytokine levels from patients on a phase 1 clinical trial (NCT03037645) testing vecabrutinib in B cell malignancies also were measured at baseline and four weeks after treatment. It was found that vecabrutinib significantly reduced the levels of multiple pro-inflammatory cytokines linked to CAR T cell toxicity, including MIP-1β, IP-10 and TNF-α (FIGS. 3A-C), among patients that maintained a stable disease, further confirming the findings described above. Taken together, the results provided herein indicate that BTK inhibitors that inhibit BTK polypeptide activity via non-covalent binding such as vecabrutinib can decrease CAR T therapy associated toxicities without affecting key CAR T cell functions.

### Reversible BTK Inhibition in Vivo Enhances Car T Cell Antitumor Activity in Xenograft Models

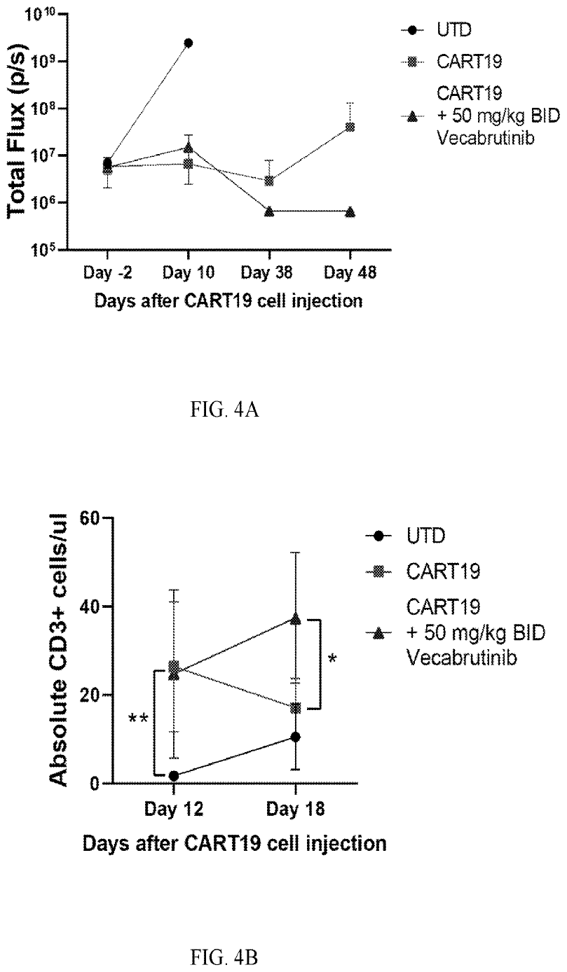

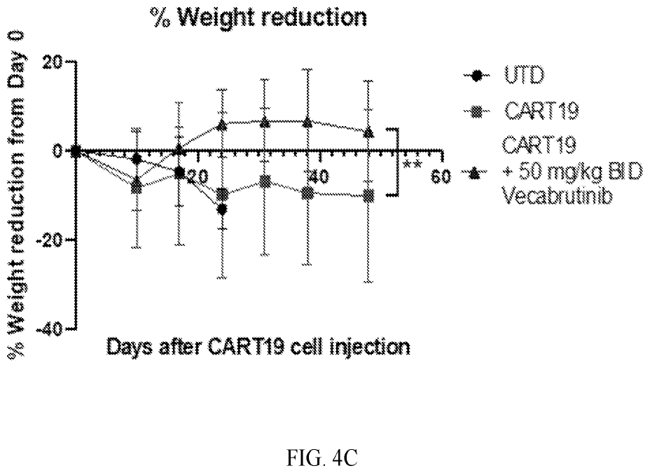

To confirm that BTK inhibitors that inhibit BTK polypeptide activity via non-covalent binding such as vecabrutinib do not inhibit CART19 cell effector functions, vecabrutinib's role on CART19 cell antitumor activity was assessed in xenograft models. NOD/SCID/IL-2 receptor y null (NSG) mice were injected with 1 million luciferase⁺ JeKo-1 cells and imaged 14 days later, allowing enough time for tumor engraftment. Mice were then randomized to receive a single injection of CART19 or UTD T cells. The CART19 group was further randomized to one group receiving vehicle or vecabrutinib via oral gavage for 30 days. Bioluminescence imaging 1 week after CART19 cell injection showed that CART19 in combination with vehicle or vecabrutinib effectively controlled the lymphoma in this tumor model (FIG. 4A). The UTD group at that point had a significant tumor burden necessitating euthanasia (FIG. 4A). Tumor control was maintained in the mice receiving vecabrutinib; however, there was a trend for early recurrence in the CART19 group treated with vehicle control (FIG. 4A). This coincided with significantly higher levels of CART19 present in the serum of vecabrutinib-treated mice, as measured by flow cytometry (FIG. 4B). The CD3⁺ cells reflect the CART cells. Mice treated with vecabrutinib better maintained their body weight following CART19 infusion compared to those treated with vehicle, suggesting possibly dampened toxicity (FIG. 4C). These results indicate that BTK inhibitors that inhibit BTK polypeptide activity via non-covalent binding such as vecabrutinib can be used in combination with CART19 treatment to lead to sustained antitumor activity and enhanced CART persistence.

### Vecabrutinib Induces Transcriptomic Changes Enhancing Proliferation of CART19 Cells

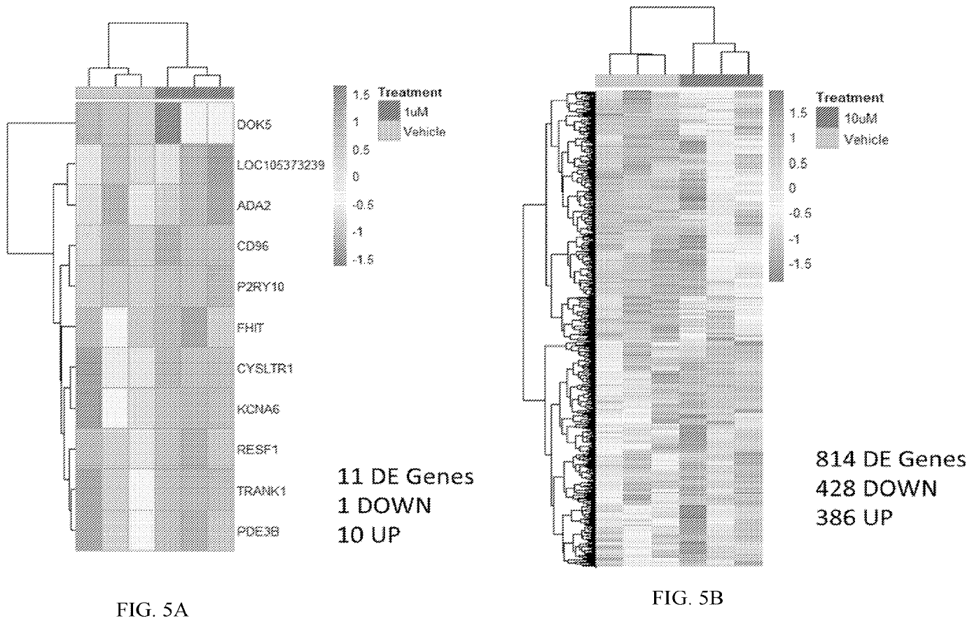

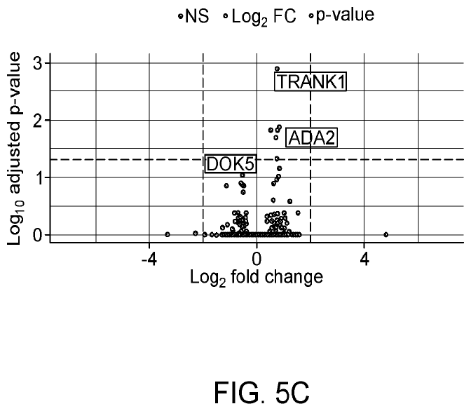

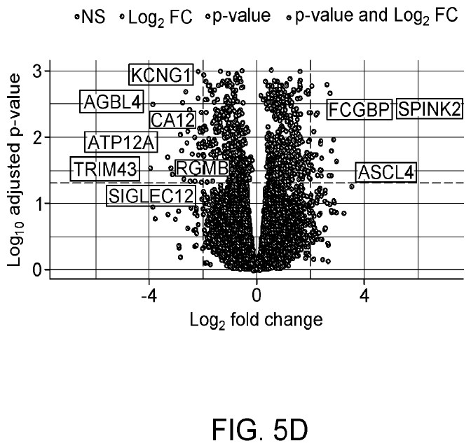

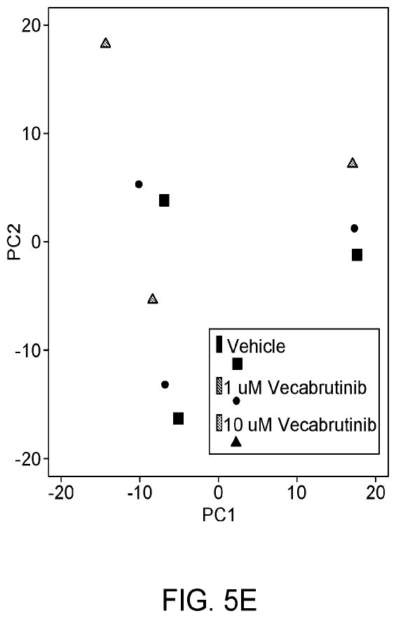

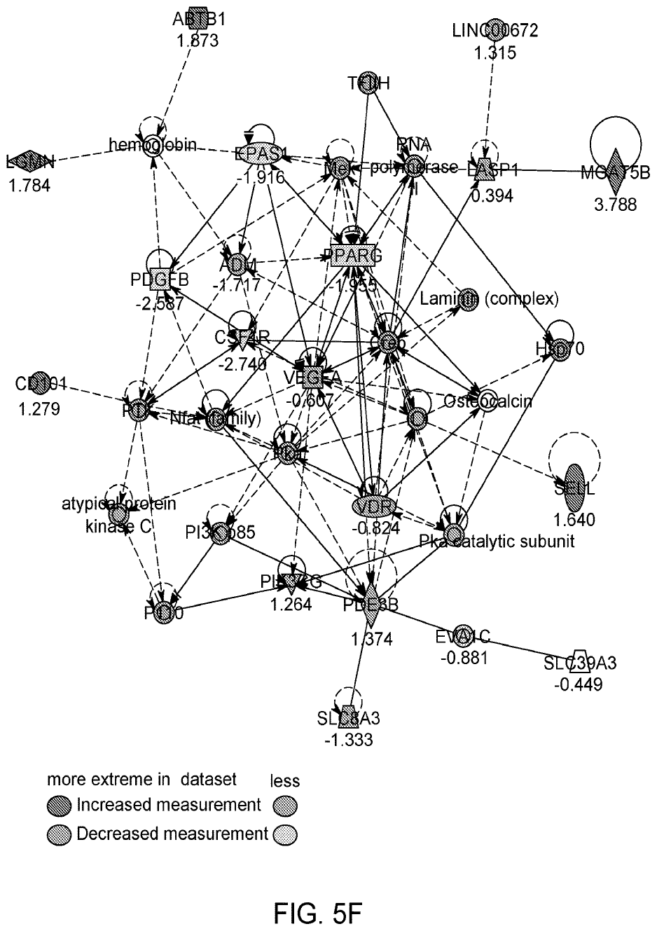

To further investigate the mechanism of vecabrutinib-induced CART19 modulation, the transcriptome of stimulated CART19 cells was interrogated in the presence of absence of 10 μM vecabrutinib. CART19 cells were stimulated through the CAR by co-culturing with irradiated JeKo-1 cells. Total RNA sequencing (RNA-Seq) of activated CART19 cells highlighted significant transcriptomic changes between cells exposed to vehicle versus those exposed to vecabrutinib (FIG. 5A). Ingenuity pathway analysis predicted increased activation of the PI3K/AKT pathway in CART19 cells co-cultured with vecabrutinib (FIG. 5B). These findings suggest that vecabrutinib can induce transcriptional hallmarks of T cell proliferation.

Taken together, these results demonstrate that BTK inhibitors that inhibit BTK polypeptide activity via non-covalent binding such as vecabrutinib can be used to modulate CAR T cell (e.g., CART19) functions by increasing their efficacy and decreasing toxicity, while maintaining their proliferative potential. Accordingly, a reversible BTK inhibitor such as vecabrutinib can be used in combination with CAR T cell therapy to increase CAR T cell efficacy and to decrease CAR T cell toxicity.

### Example 2: Mechanisms of Vecabrutinib-Induced Cart19 Modulation

### Experimental Details

Biological Replicates: C267, C278, C296 where CART19 cells were generated
Conditions:
   Vehicle
   1 μM vecabrutinib
   10 μM vecabrutinib
Activated for 72 hours with Irradiated JeKo-1 (1:1 E:T Ratio)
mRNA was extracted from CART19 cells
RNA sequencing was performed

### Analysis Pipeline on RNA Seq Data

FastQC (initial quality check)
Trimmomatic (trim adapters and overrepresented sequences)
FastQC (to confirm removal of the above)
STAR (align to the latest human genome annotation, GRCh38.p13)
HTSeq (obtain counts for each gene)
DESeq2
   Normalized counts across samples
   Calculated differential expression between groups
   Multiple hypothesis correction (Benjamini-Hochberg procedure)

### Results

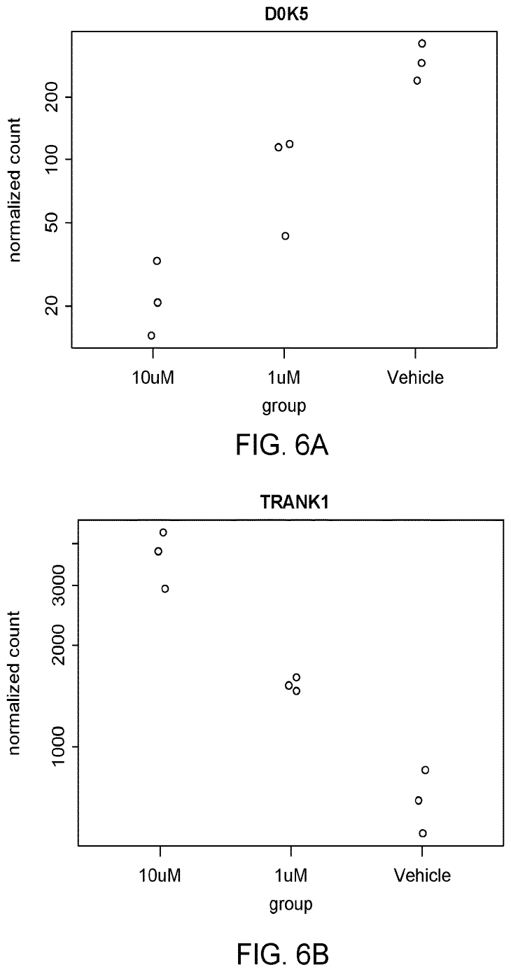

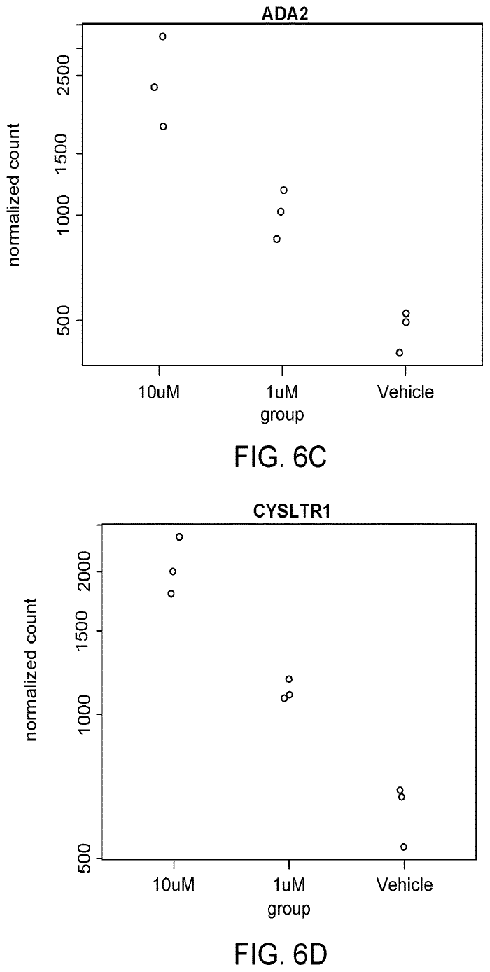

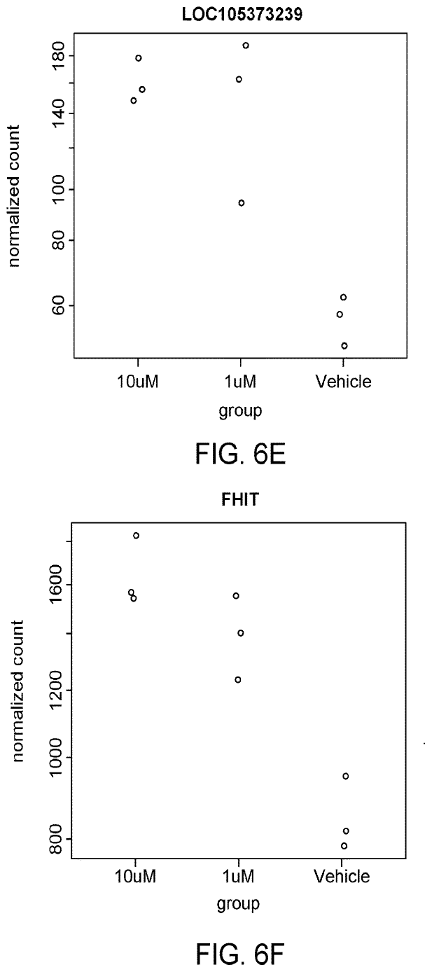

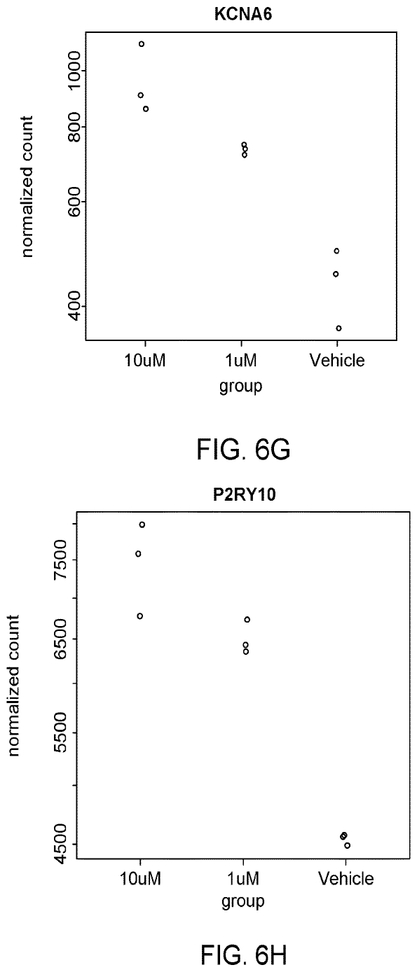

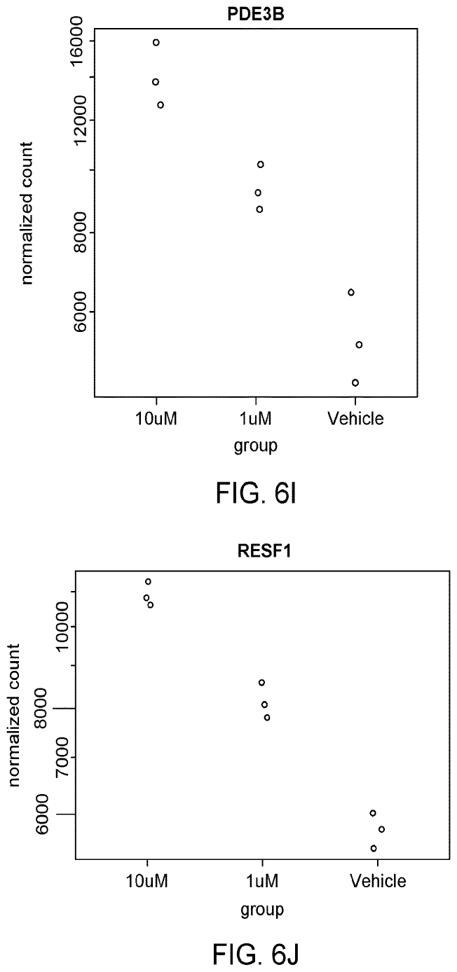

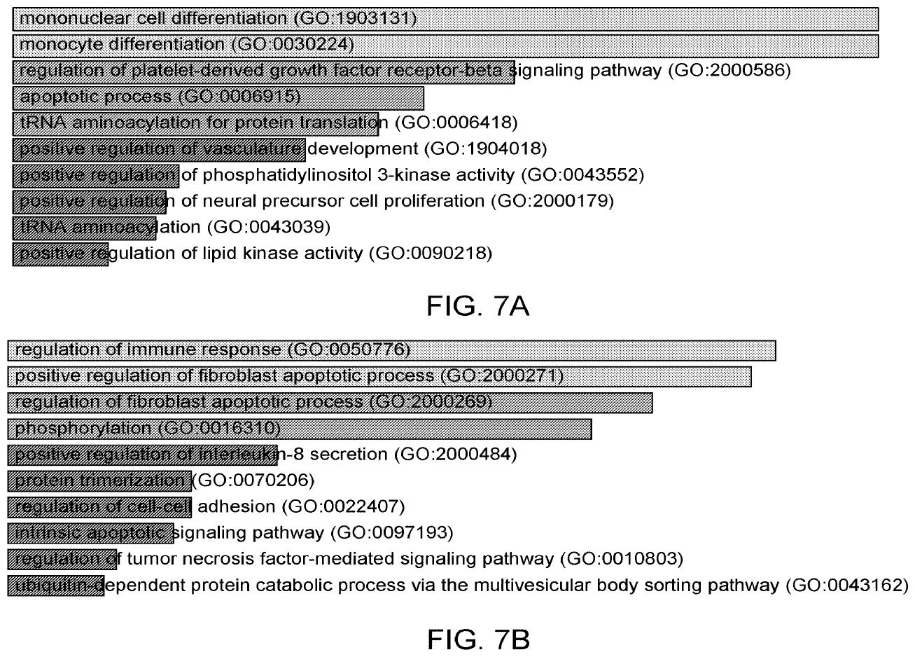

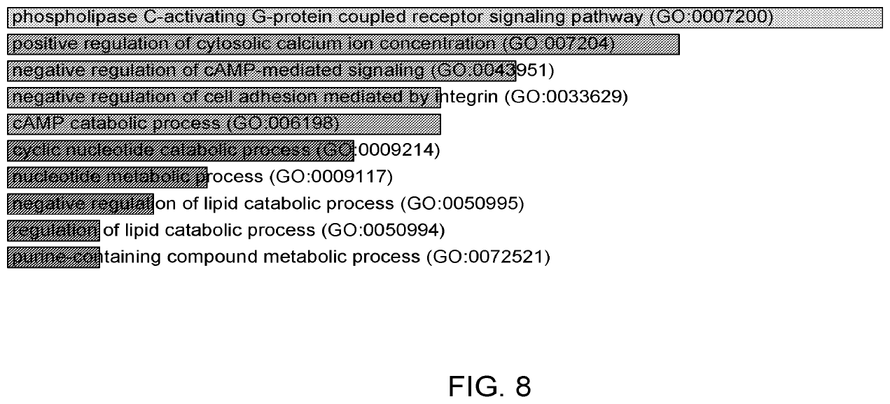

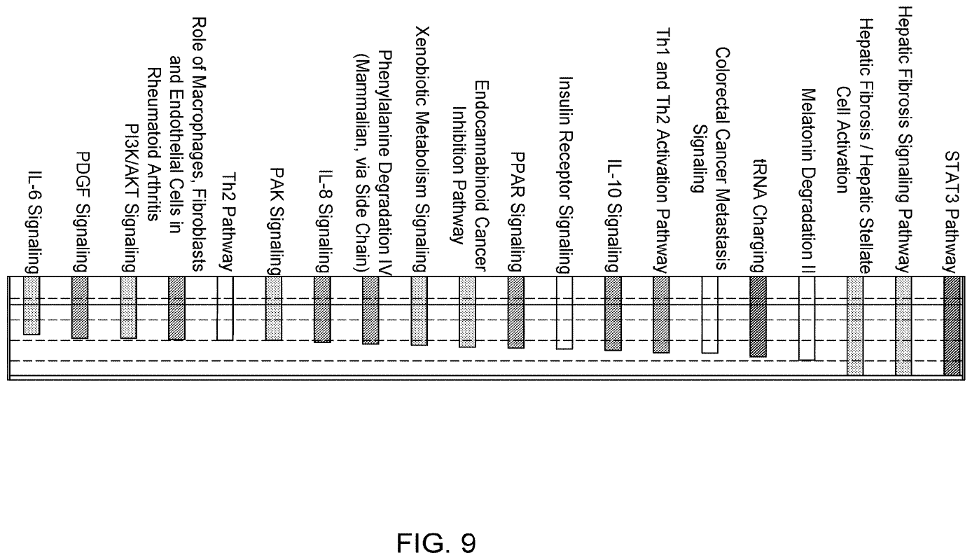

CART19 cells were co-cultured with irradiated JeKo-1 in the presence of vehicle (DMSO) or vecabrutinib for 72 hours via RNA-Seq. Differentially expressed genes are shown in FIG. 6. Multiples pathways are enriched in CART19 cells that are co-cultured with vecabrutinib. Differentially expressed genes involved in a GSEA biological process are shown in FIG. 7 and FIG. 8. Differentially expressed genes involved canonical pathways from ingenuity pathway analysis are shown in FIG. 9.

TH 1/TH2 activation pathway is enriched. p-value=2.62E-04. TH2 pathway is enriched. p-value=1.01E-03. TH1 pathway is enriched. Positive Z-score (0.816). p-value=4.19E-03.
PTEN signaling pathway. Z-score 0.333. p-value 1.68E-02
STAT 3 pathway is enriched. Negative z-score (−3.051). p-value=1.57E-07.
IL-10 signaling pathway is enriched. p-value=3.3E-04.
IL-8 signaling pathway is enriched. Negative z-score (−1.941). p-value=7.86E-04.
PI3K/AKT signaling is enriched. Positive z-score (0.707). p-value=1.2E-03.
PDGF signaling is enriched. Negative z-score (−1.667). p-value=1.22E-03.

### Example 3: Comparison Between Ibrutinib and Vecabrutinib

### Cytotoxicity

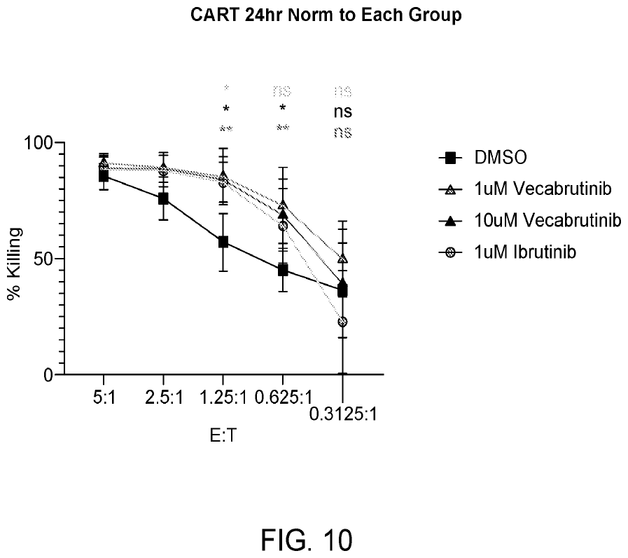

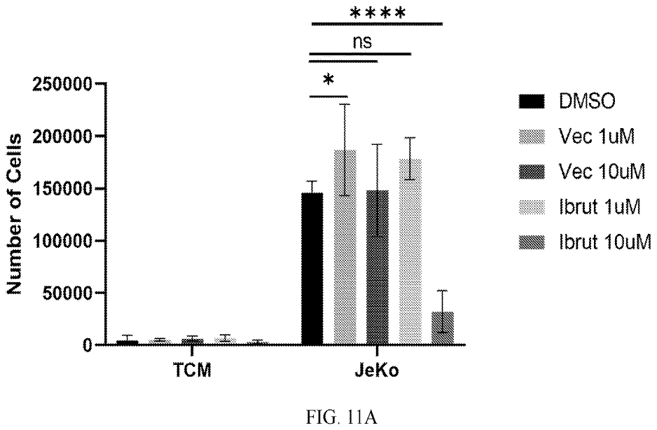

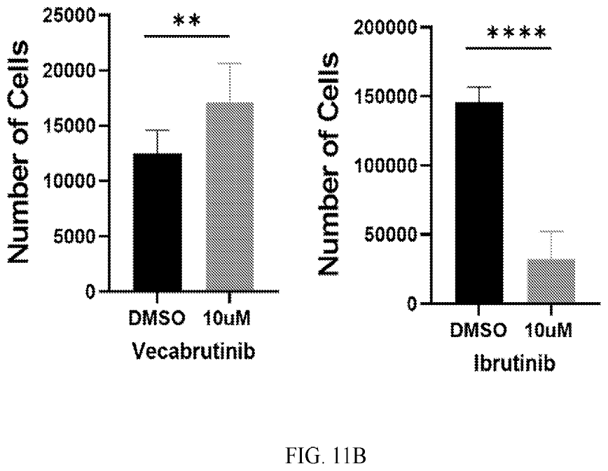

Measurement of CART19 killing of JeKo-1 cells at different effector to target ratios. At a 0.625:10 ratio, vecabrutinib significantly potentiated CART 19-mediated killing of Jeko-1 lymphoma cells, whereas ibrutinib had no effect (FIG. 10). Results were normalized to target cells incubated with the respective concentration of the BTK inhibitor alone (with no CART19) to eliminate the direct cytotoxic effect of the inhibitor on lymphoma cells without CART19. 10 μM of ibrutinib was not included as it caused 100% killing of CART19 cells and Jeko-1 cells (FIGS. 11A and 11B).

### Proliferation

Measurement of CART19 proliferation induced by irradiated JeKo-1 lymphoma cells in the presence of different BTK inhibitors. Vecabrutinib enhanced CART19 proliferation (FIGS. 11A and 11B). 1 μM of ibrutinib had no effect on CART19 proliferation (FIG. 11A), whereas 10 μM of ibrutinib was toxic (FIGS. 11A and 11B).

### Effect of BTK Inhibitors on Cytokine and Chemokine Secretions

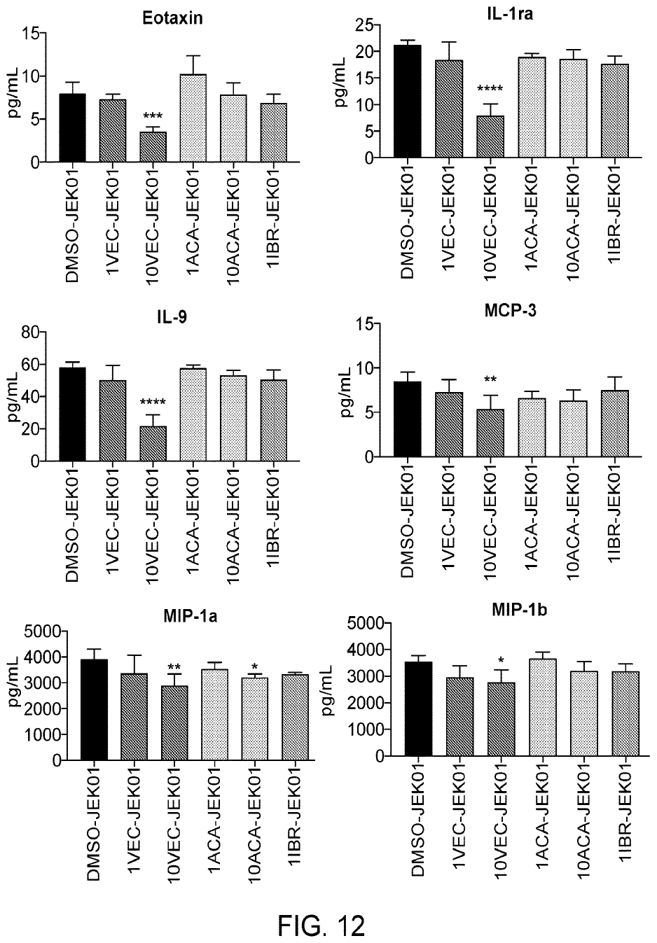

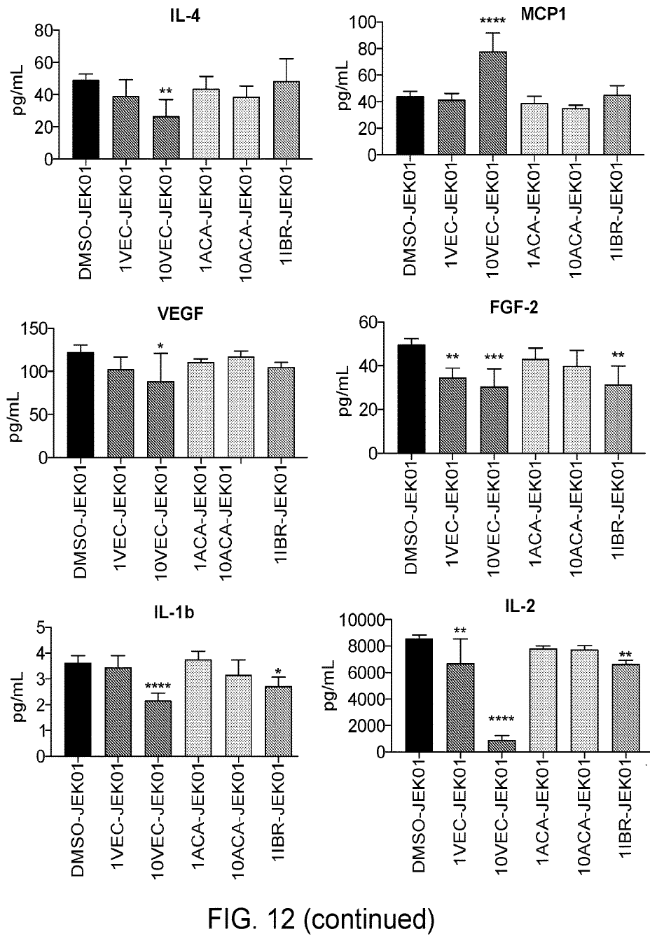

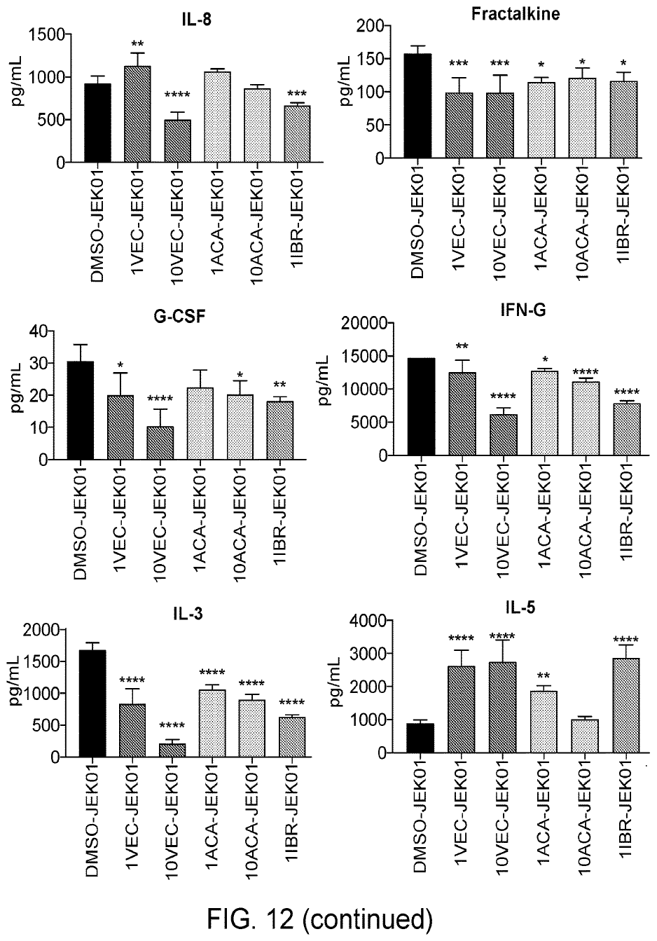

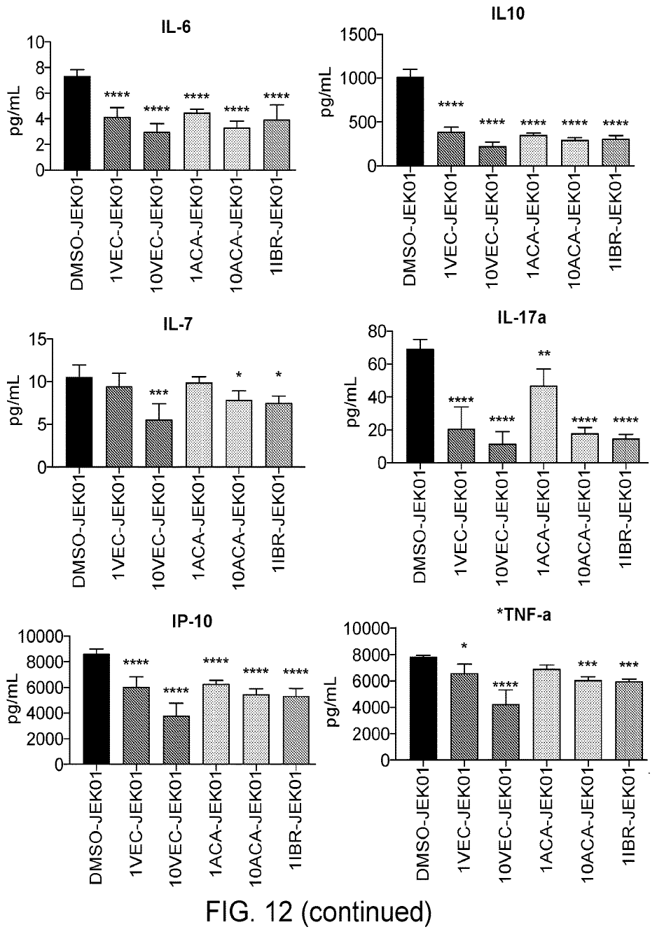

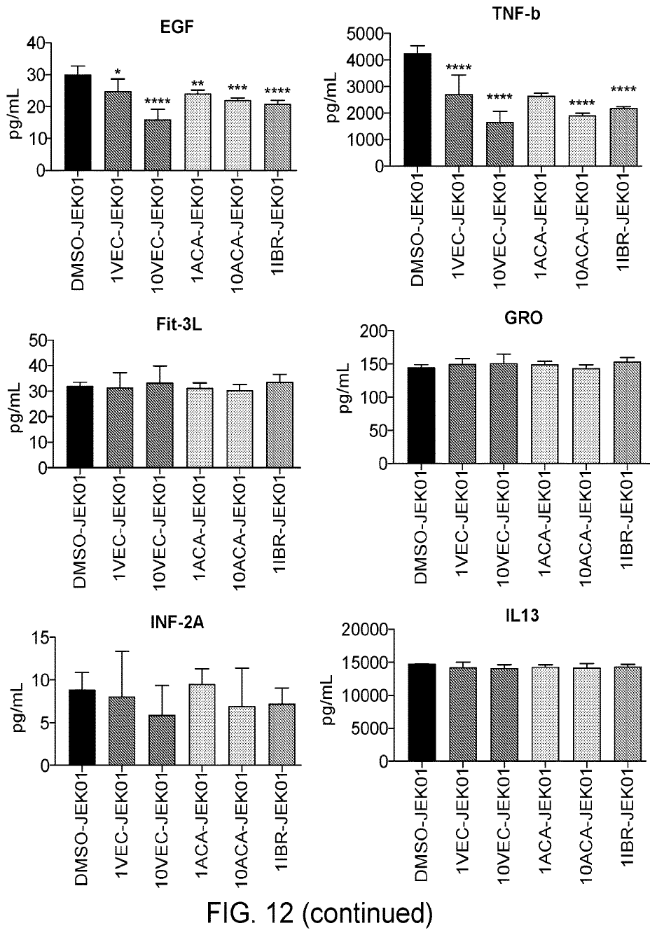

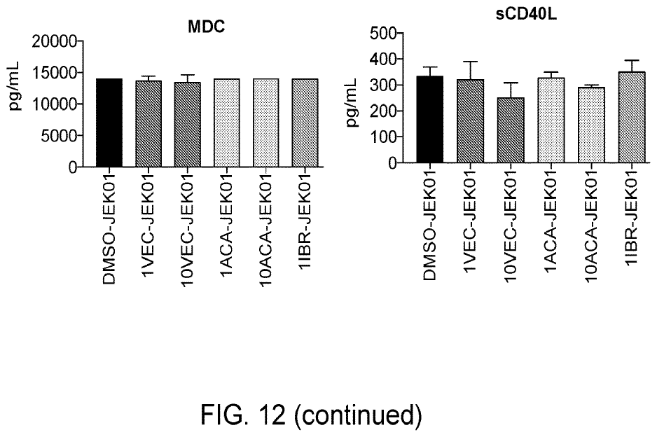

CART19 and JeKo-1 were co-cultured at 1:1 ratio for 24 hours in the presence of DMSO, vecabrutinib, vecabrutinib, acalabrutinib, acalabrutinib (10ACA-JEKO1), or ibrutinib (FIG. 12).

### Example 4: Treating Cancer with CAR T cells and a BTK inhibitor that inhibits BTK Polypeptide Activity Via Non-Covalent Binding

CAR T cells (e.g., CART19 cells) are systemically administered to a mammal (e.g., a human) having cancer, and a BTK inhibitor that inhibits BTK polypeptide activity via non-covalent binding (e.g., vecabrutinib) is orally or systemically administered to that mammal. The administered BTK inhibitor (e.g., vecabrutinib) can result in an improved CAR T cell antitumor activity and/or enhanced CAR T cell persistence.

### Example 5: Treating Cancer with CAR T cells and a BTK inhibitor that Inhibits BTK Polypeptide Activity Via Non-Covalent Binding

One or more BTK inhibitors (e.g., vecabrutinib) are orally or systemically administered to a mammal (e.g., a human) having cancer and scheduled to receive an adoptive T cell therapy (e.g., a CAR T cell therapy) to treat the cancer. Thereafter, the mammal is systemically administered an adoptive T cell therapy (e.g., a CAR T cell therapy).

The administered BTK inhibitor (e.g., vecabrutinib) can result in an improved anti-cancer activity of the adoptive T cells (e.g., CAR T cells) and/or enhanced adoptive T cell (e.g., CAR T cell) persistence.

### Example 6: Treating Cancer with Cart Cells and a BTK Inhibitor that Inhibits BTK Polypeptide Activity Via Non-Covalent Binding

One or more BTK inhibitors (e.g., vecabrutinib) are orally or systemically administered to a mammal (e.g., a human) having cancer and having received an adoptive T cell therapy (e.g., a CART cell therapy) to treat the cancer.

The administered BTK inhibitor (e.g., vecabrutinib) can result in an improved anti-cancer activity of the adoptive T cells (e.g., CAR T cells) and/or enhanced adoptive T cell (e.g., CAR T cell) persistence.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for improving T cell therapy, wherein said method comprises administering, to a mammal and at a time that is from about 1 day before to about 360 days after said mammal receives an adoptive T cell therapy, a reversible BTK inhibitor that binds non-covalently to a BTK polypeptide, wherein the level of proliferation of T cells of said adoptive T cell therapy in said mammal is increased after said administering step as compared to the level of proliferation of T cells of an adoptive T cell therapy in a comparable mammal not administered said BTK inhibitor.

2. The method of claim 1, wherein said level of proliferation is increased by at least 5 percent.

3. (canceled)

4. (canceled)

5. A method for improving T cell therapy, wherein said method comprises administering, to a mammal and at a time that is from about 1 day before to about 360 days after said mammal receives an adoptive T cell therapy, a reversible BTK inhibitor that binds non-covalently to a BTK polypeptide, wherein said adoptive T cell therapy is less toxic to said mammal than the level of toxicity in a comparable mammal receiving an adoptive T cell therapy and not administered said BTK inhibitor.

6. A method for improving T cell therapy, wherein said method comprises administering, to a mammal and at a time that is from about 1 day before to about 360 days after said mammal receives an adoptive T cell therapy, a reversible BTK inhibitor that binds non-covalently to a BTK polypeptide, wherein T cells of said adoptive T cell therapy in said mammal express a reduced level of one or more polypeptides after said administering step as compared to the level of said one or more polypeptides expressed by T cells of an adoptive T cell therapy in a comparable mammal not administered said BTK inhibitor, wherein said one or more polypeptides are selected from the group consisting of an MIP-la polypeptide, an MIP-1β polypeptide, a TNF-α polypeptide, a TNF-β polypeptide, an eotaxin polypeptide, an MCP-3 polypeptide, an IP-10 polypeptide, a VEGF polypeptide, an EGF polypeptide, a G-CSF polypeptide, an IFN-γ polypeptide, an FGF-2 polypeptide, a fractalkine polypeptide, an IL-1ra polypeptide, an IL-1β polypeptide, an IL-2 polypeptide, an IL-3 polypeptide, an IL-4 polypeptide, an IL-6 polypeptide, an IL-7 polypeptide, an IL-9 polypeptide, an IL-10 polypeptide, and an IL-17A polypeptide.

7. The method of claim 6, wherein said one or more polypeptides are said MIP-1β polypeptide, said IP-10 polypeptide, and said TNF-α polypeptide.

8. The method of claim 6, wherein said one or more polypeptides are said MIP-1β polypeptide, said TNF-α polypeptide, said IP-10 polypeptide, said IL-4 polypeptide, said IL-6 polypeptide, and said IL-10 polypeptide.

9. The method of claim 6, wherein said one or more polypeptides are said MIP-1α polypeptide, said MIP-1β polypeptide, said eotaxin polypeptide, said MCP-3 polypeptide, said VEGF polypeptide, said IL-1ra polypeptide, said IL-4 polypeptide, and said IL-9 polypeptide.

10. The method of claim 6, wherein said one or more polypeptides are said MIP-1α polypeptide, said MIP-1β polypeptide, said TNF-α polypeptide, said TNF-β polypeptide, said eotaxin polypeptide, said MCP-3 polypeptide, said IP-10 polypeptide, said VEGF polypeptide, said EGF polypeptide, said G-CSF polypeptide, said IFN-γ polypeptide, said FGF-2 polypeptide, said fractalkine polypeptide, said IL-1ra polypeptide, said IL-1β polypeptide, said IL-2 polypeptide, said IL-3 polypeptide, said IL-4 polypeptide, said IL-6 polypeptide, said IL-7 polypeptide, said IL-9 polypeptide, said IL-10 polypeptide, and said IL-17A polypeptide.

11. A method for improving T cell therapy, wherein said method comprises administering, to a mammal and at a time that is from about 1 day before to about 360 days after said mammal receives an adoptive T cell therapy, a reversible BTK inhibitor that binds non-covalently to a BTK polypeptide, wherein T cells of said adoptive T cell therapy in said mammal express an increased level of one or more polypeptides after said administering step as compared to the level of said one or more polypeptides expressed by T cells of an adoptive T cell therapy in a comparable mammal not administered said BTK inhibitor, wherein said one or more polypeptides are selected from the group consisting of an MCP-1 polypeptide and an IL-5 polypeptide.

12. (canceled)

13. A method for improving T cell therapy, wherein said method comprises administering, to a mammal and at a time that is from about 1 day before to about 360 days after said mammal receives an adoptive T cell therapy, a reversible BTK inhibitor that binds non-covalently to a BTK polypeptide, wherein T cells of said adoptive T cell therapy in said mammal have an increased percentage of target cell killing after said administering step as compared to the percentage of target cell killing by T cells of an adoptive T cell therapy in a comparable mammal not administered said BTK inhibitor.

14. The method of claim 13, wherein said percentage of target cell killing is increased by at least 5 percent.

15. (canceled)

16. (canceled)

17. The method of claim 1, wherein said mammal is a human.

18. The method of claim 1, wherein said mammal has cancer, and wherein said adoptive T cell therapy treats said cancer following said administering step.

19. The method of claim 18, wherein said cancer is lymphoma, leukemia, hepatocellular carcinoma, bowel cancer, lung cancer, breast cancer, ovarian cancer, melanoma, head and neck cancer, sarcoma, epithelial cancer, mesothelioma, thyroid cancer, brain cancer, or endocrine cancer.

20. The method of claim 1, wherein said mammal has lymphoma, and wherein said adoptive T cell therapy treats said lymphoma following said administering step.

21. The method of claim 1, wherein said BTK inhibitor is selected from the group consisting of vecabrutinib, fenebrutinib, pirtobrutinib, ARQ531, XMU-MP3, CB1763, GNE-431, CGI-1746, RN1486, BMS-986142, HBW-3-10, and CG806.

22. The method of claim 1, wherein said T cells of said adoptive T cell therapy comprise a chimeric antigen receptor.

23. The method of claim 22, wherein said chimeric antigen receptor targets a tumor-associated antigen.

24. The method of claim 23, wherein said tumor-associated antigen is CD19.

25. The method of claim 1, wherein said adoptive T cell therapy is a CAR T therapy.

26. The method of claim 1, wherein said adoptive T cell therapy causes less expression of one or more cytokine polypeptides associated with cytokine release syndrome within said mammal than the level of expression of said one or more cytokine polypeptides within said comparable mammal that received said adoptive T cell therapy in the absence of said administration of said BTK inhibitor.

27. The method of claim 26, wherein said cytokine polypeptides are selected from the group consisting of an IL-6 polypeptide, an IL-10 polypeptide, an MIP-β polypeptide, an IP-10 polypeptide, and a TNF-α polypeptide.

28. The method of claim 1, wherein said BTK inhibitor is administered to said mammal at a dose from about 10 mg/kg to about 250 mg/kg.

29-32. (canceled)
